(19) United States
(12) Patent Application Publication
(10) Pub. No.: US 2007/0055383 A1
(43) Pub. Date: Mar. 8, 2007

(54) Vacuum assisted heat/perspiration removal system and limb volume management for prosthetic device

(76) Inventor: Charles King, Cumberland, MD (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(21) Appl. No.: 11/518,064

(22) Filed: Sep. 8, 2006

Related U.S. Application Data
(60) Provisional application No. 60/715,313, filed on Sep. 8, 2005.
(60) Provisional application No. 60/724,512, filed on Oct. 8, 2005.
(60) Provisional application No. 60/749,942, filed on Dec. 12, 2005.
(60) Provisional application No. 60/759,327, filed on Jan. 14, 2006.
(60) Provisional application No. 60/760,074, filed on Jan. 18, 2006.
(60) Provisional application No. 60/760,596, filed on Jan. 21, 2006.
(60) Provisional application No. 60/777,240, filed on Feb. 27, 2006.
(60) Provisional application No. 60/798,533, filed on May 8, 2006.
(60) Provisional application No. 60/833,368, filed on Jul. 26, 2006.
(60) Provisional application No. 60/837,805, filed on Aug. 14, 2006.

(51) Int. Cl. A61F 2/80

(52) U.S. Cl. 623/34

(57) ABSTRACT

The vacuum assisted liner system is for use with a prosthetic device to be attached to a residual limb. The liner system includes a hypobaric prosthetic liner, and a porous wicking material layer to surround at least a portion of the residual limb and define a regulated vacuum environment between the hypobaric prosthetic liner and the residual limb. The hypobaric prosthetic liner has at least one passageway therethrough defining at least one vacuum port, such as an inlet port and outlet port, in fluid communication with the regulated vacuum environment. Internal liner passageways may connect the inlet and outlet ports to the regulated vacuum environment. A vacuum regulation device may include an electric vacuum pump or a motion activated pump connected to the outlet port.

### CROSS-REFERENCE TO RELATED APPLICATIONS

This application claims the benefit of U.S. Provisional Application Nos. 60/715,313 filed Sep. 8, 2005, 60/724,512 filed Oct. 8, 2005, 60/749,942 filed Dec. 12, 2005, 60/759,327 filed Jan. 14, 2006, 60/760,074 filed Jan. 18, 2006, 60/760,596 filed Jan. 21, 2006, 60/777,240 filed Feb. 27, 2006, 60/798,533 filed May 08, 2006, 60/833,368 filed Jul. 26, 2006 and 60/837,805 filed Aug. 14, 2006, all of which are hereby incorporated herein in their entireties by reference.

### FIELD OF THE INVENTION

The present invention relates to the field of prosthetics, and, more particularly, to prosthetic device socket liners, related systems and related methods.

### BACKGROUND OF THE INVENTION

Excessive heat, perspiration and daily residual limb volume fluctuations are problems encountered by the amputee population wearing a prosthetic liner and limb. The prosthetic liner, which is donned upon the residual limb of the amputee, for both suspension and alleviation of shear and pressure on the residual limb, can be described as a non-porous elastomeric material with high thermal insulation properties. A prosthetic liner seals off air flow to the residual limb, which results in heat and perspiration build up. Because of these factors and especially in hot climates the skin of the residual limb becomes susceptible to infections, allergies and other skin diseases. Perspiration decreases the friction between the residual limb and the liner. This can cause a pistoning action between the liner and limb that macerates the skin, as well as create the potential for catastrophic failure of the suspension of the limb.

Daily volume loss is also a widespread problem of amputees. A study conducted by W. Board, G. Street and C. Caspers, entitled A comparison of Trans-Tibal Amputee Suction and Vacuum Socket Conditions (Prosthetic and Orthotics International, 2001, 25) found that on average the residual limb volume of the test subjects decreased by 6.5 & during only a thirty minute walk (page 205). Volume loss leads to diminished proprioception, poor fit of the socket and discomfort. Bony prominences can also experience higher shear and pressure because of the change in the residual limb volume.

There are prior art examples of an elevated vacuum being applied directly to the skin of the amputee. For example, U.S. Pat. No. 5,888,230 and U.S. Pat. No. 6,231,616 both to Helmy and both described as MODULAR LINER FOR LIMB RESIDUAL LIMB PROSTHESIS. These patents describe the use of vacuum to remove interstitial spacing or gapping in a prosthetic liner; an even application of vacuum is not distributed over the residual limb. The liner inherently seals locally to the vacuum source in contact with the skin, given the pliable nature of both.

U.S. Pat. No. 6,974,484 to Caspers and described as OSMOSTIC MEMBRANE and VACUUM SYSTEM for ARTIFICIAL LIMB specifically details the use of an osmotic membrane for perspiration removal from an artificial limb. An osmotic membrane is a selectively permeable membrane “that allows water vapor to pass from the limb but prevents liquid from passing to the limb.” Although U.S. Pat. No. 6,974,484 uses an osmotic membrane, it does not allow the inflow of air while maintaining a vacuum between limb and liner.

In the prior art there is an example of a prosthetic liner fitted with a check valve. U.S. Pat. No. 6,544,292 to Laghi describes a device as a PROSTHETIC LINER WITH INTEGRAL AIR EXPULSION VALVE. This patent depicts a prosthetic liner with an air expulsion valve built into the walls of the liner, to facilitate donning of the liner and creating an airtight seal upon the limb. There is no mention of perspiration removal or its application with elevated vacuum. Also, U.S. Pat. No. 5,728,169 to Norvell details a moisture retention prevention interface between the limb and prosthetic liner. There is no mention of the vacuum source or of any mass air flow mechanism in this patent.

U.S. Patent Application Publication No. 2004/0167447 to Johnson describes a device as an Orthopedic Appliance with Moisture Management System. This application shows the use of a fabric liner “serving to wick away moisture directly through. . . vent holes.” There is no mention of elevated vacuum creating a mass air flow mechanism in this patent. U.S. Patent Application Publication No. 2005/0197611 Taranow described as a VACUUM-SEALED ORTHOTIC, PROSTHETIC, and OTHER BODY WORN DEVICES details designs that provide “active suctioning” of “evacuatable sleeve” used for suspension of a “artificial foot or leg” or “artificial hand or arm.” The design is basically for a sleeve suspension of an artificial limb that sub-atmospheric pressure is applied to. There is no mention of perspiration removal, or any means of maintaining the vacuum inside the suspension sleeve once evacuated.

U.S. Pat. No. 6,726,726 to Caspers described as a VACUUM APPARATUS AND METHOD FOR MANAGING RESIDUAL LIMB VOLUME IN AN ARTIFICIAL LIMB, details the trouble with “edema and blistering at the point on the residual limb where the suspension sleeve contacts the residual limb.” Given the configuration detailed in this patent, unregulated vacuum was being exposed to skin of the amputee above the liner and beneath the sealing sleeve. Because of the gap that formed from the thickness of the liner, and a sleeve rolled over it and up on the thigh, edema and blistering occurred when unregulated vacuum was applied to the created void. This passage is cited because this was a potential mechanism that perspiration could be pulled from between the limb and liner, migrating up from the proximal aspect of the liner. The patent details several designs to prevent this occurrence but in practice by the manufacturer, the liner was made longer, extended up on the thigh preventing the sealing sleeve from extending beyond the proximal aspect of the liner, ultimately stopping unregulated vacuum from being exposed to the skin of the amputee.

Limb volume management is described in the study entitled, A Comparison of Trans-Tibial Amputee Suction and Vacuum Socket Conditions, by W. Board, G. Street, and C. Caspers published in Prosthetics and Orthotics International, 2001; vol. 25, 202-209.

Suction suspension of an artificial limb is a standard accepted prosthetic design protocol. An example of the prior art for a prosthetic suction valve is U.S. Pat. No. 2,834,025 (Leavy , Jerry D.) for a device described as SUCTION DEVICE FOR PROSTHETIC LIMB [1958]. This patent describes a one-way valve which can be disassembled so that a donning sheath can be pulled through its opening and reassembled maintaining the suction or vacuum seal. Suction suspension of this sort can be classified as non-elevated vacuum suspension and is employed throughout prosthetic practice, in limbs with or without prosthetic liners.

It has long been established in clinical practice that suction suspension is optimal for certain levels of amputation. The observed benefits are control of edema, improved circulation, improved control of the prosthesis, and increased proprioception. It was until very recently that suction suspension implied non-elevated vacuum suspension.

Elevated vacuum via mechanical or electronic vacuum pumps is a relatively new, but accepted, practice. As a measure of the new general acceptance of the use of these devices, the Federal Government, through its Medicare administration, effective Jan. 1, 2003, has assigned specific L-codes to prosthetic vacuum pumps, L5781 and L5782 respectively. L5781 reads: Addition to lower limb prosthesis, vacuum pump, residual limb volume management and moisture evacuation system (L5782 is for a higher weight capacity vacuum pump).

It should be noted that none of the commercially available vacuum pumps have ever reliably achieved moisture evacuation in a prosthetic socket. U.S. Pat. No. 6,726,726 to Caspers, Carl A. for a device detailed as VACUUM APPARATUS AND METHOD FOR MANAGING RESIDUAL LIMB VOLUME IN AN ARTIFICIAL LIMB is a continuation of a series of patents for a popular prosthetic vacuum pump. In the description of the preferred embodiment, it is revealed that “the vacuum which holds the residual limb (and liner) in firm contact with the socket tends to cause edema and blistering at the point on the residual limb where the suspension sleeve contacts the residual limb.” This is the only area for moisture to evacuate from the liner and it was initially believed to be a viable way for perspiration control, as evidenced by the description of the Medicare L-Code. When reduced to practice, unregulated vacuum applied to human skin caused breakdowns as described above. If regulated vacuum was applied to this area, effective perspiration removal would not occur because of the inefficiency of the design.

Prosthetic liners are prevalent throughout prosthetics. An example of the prior art for a method of constructing a pliable prosthetic liner is found in U.S. Pat. No. 3,377,416 to Krandel described as METHOD OF MAKING LINER FOR ARTIFICIAL LIMB. This patent describes the fabrication method of creating a R.T.V. rubber prosthetic liner, whose “support is obtained from the entire stump surface.” This foreshadows the coming acceptance and use of total surface bearing prosthetic liners, where the load and shear forces of a prosthetic socket are distributed evenly over the flexible liner, which is donned upon the amputee's residual limb.

A problem with a total surface bearing (TSB) socket and liner can be found in the abstract of U.S. Pat. No. 5,258,037 to Caspers for a device detailed as PROSTHETIC LINER AND METHOD OF MAKING THE LINER WITH A PROSTHESIS SOCKET. This patent describes a liner that is tight on the amputee's residual limb and big for the receiving socket. Upon donning the liner and wearing the leg, compressive loads force interstitial fluid from the limb and accelerate shrinkage, and atrophy of the limb is the unfortunate characteristic for a total surface bearing liner reduced to prosthetic practice.

There is a need for an approach to immobilize the skin, reducing relative motion, which transfers load and shear to the liner. The approach should contribute to perspiration control, cooling of the residual limb, and improve suspension by limiting pistoning, and assist in daily volume management of the residual limb of an amputee.

### SUMMARY OF THE INVENTION

In view of the foregoing background, it is therefore an object of the present invention to provide a liner, system and method to immobilize the skin reducing relative motion which transfers load and shear to the liner, control perspiration, cool the residual limb, improve suspension by limiting pistoning, and assist in daily volume management of the residual limb of an amputee.

This and other objects, features, and advantages in accordance with the present invention may be provided by a vacuum assisted liner system for use with a prosthetic device to be attached to a residual limb. The liner system includes a hypobaric prosthetic liner to be donned over the residual limb, and a porous wicking material layer to surround at least a portion of the residual limb and define a regulated vacuum environment between the hypobaric prosthetic liner and the residual limb. The hypobaric prosthetic liner has at least one passageway therethrough defining at least one vacuum port in fluid communication with the regulated vacuum environment.

The hypobaric prosthetic liner may include a sealing apron adjacent a proximal end thereof to create a seal between the hypobaric prosthetic liner and at least a portion of the prosthetic device. Also, the porous wicking layer may be attached to the hypobaric prosthetic liner. The at least one vacuum port preferably includes an inlet port in fluid communication with the regulated vacuum environment and an outlet port in fluid communication with the regulated vacuum environment.

The at least one passageway preferably includes a first internal liner passageway connecting the inlet port to the regulated vacuum environment, and a second internal liner passageway connecting the outlet port to the regulated vacuum environment. A vacuum regulation device may be connected at least to the outlet port, and may include an electric vacuum pump connected to the outlet port, and an associated controller. Furthermore, the vacuum regulation device may include a flow control switch, such as a solenoid valve, connected to the inlet port.

The vacuum regulation device may also include a battery to power the vacuum pump, controller and solenoid valve, and a housing may contain the battery, vacuum pump, controller and solenoid valve. Also, a remote control transmitter may be included for controlling the solenoid valve. A manually adjustable vacuum relief valve may be connected to the inlet port.

The vacuum regulation device may alternatively include a motion actuated vacuum pump connected to the outlet port, and an associated mechanical linkage connecting the motion actuated vacuum pump to the prosthetic device. A non-adjustable vacuum relief valve may be connected to the inlet port of the hypobaric prosthetic liner and in fluid communication with the regulated vacuum environment.

Objects, features, and advantages in accordance with the present invention may be provided by a vacuum assisted liner system for use with a prosthetic device to be attached to a residual limb, wherein the liner system includes a hypobaric prosthetic liner, and a textile fabric layer to define a regulated vacuum environment between the hypobaric prosthetic liner and the residual limb. The hypobaric prosthetic liner has a plurality of passageways therethrough defining an inlet port and an outlet port in fluid communication with the regulated vacuum environment. A vacuum regulation device is connected between the inlet port and the outlet port, the vacuum regulation device including an electric vacuum pump connected to the outlet port, and a solenoid valve connected to the inlet port.

The plurality of passageways may comprise a first internal liner tube connecting the inlet port to the regulated vacuum environment, and a second internal liner tube connecting the outlet port to the regulated vacuum environment. The vacuum regulation device may further include a battery to power the vacuum pump, controller and solenoid valve, and a housing containing the battery, vacuum pump, controller and solenoid valve. Again, a remote control transmitter may control at least the solenoid valve.

Objects, features, and advantages in accordance with the present invention may also be provided by a method of attaching a prosthetic device to a residual limb, the method including providing a hypobaric prosthetic liner, and providing a porous wicking material layer to surround at least a portion of the residual limb and define a regulated vacuum environment between the hypobaric prosthetic liner and the residual limb. The hypobaric prosthetic liner has at least one passageway therethrough defining at least one vacuum port in fluid communication with the regulated vacuum environment. The method further includes providing a vacuum regulation device to connect at least to the outlet port.

The at least one vacuum port may include an inlet port in fluid communication with the regulated vacuum environment and an outlet port in fluid communication with the regulated vacuum environment. The at least one passageway may include a first internal liner passageway connecting the inlet port to the regulated vacuum environment, and a second internal liner passageway connecting the outlet port to the regulated vacuum environment. The vacuum regulation device may include an electric vacuum pump connected to the outlet port and/or a solenoid valve connected to the inlet port.

The vacuum regulation device may further include a battery to power the vacuum pump and solenoid valve, and a housing containing the battery, vacuum pump, and solenoid valve. The vacuum regulation device may further include a remote control transmitter for controlling at least the solenoid valve. The method may include connecting a manually adjustable vacuum relief valve to the inlet port.

The vacuum regulation device may include a motion actuated vacuum pump connected for connection to the outlet port, and an associated mechanical linkage to connect the motion actuated vacuum pump to the prosthetic device. The vacuum regulation device may further include a non-adjustable vacuum relief valve connected to the inlet port of the hypobaric prosthetic liner and in fluid communication with the regulated vacuum environment. Again, the hypobaric prosthetic liner may include a sealing apron adjacent a proximal end thereof to create a seal between the hypobaric prosthetic liner and at least a portion of the prosthetic device.

The many embodiments of the present invention decribed herein immobilize the skin reducing relative motion which transfers load and shear to the liner. The embodiments of the present invention contribute to perspiration control, cooling of the residual limb, and improving suspension by limiting pistoning and assisting in daily volume management of the residual limb of an amputee.

### BRIEF DESCRIPTION OF THE DRAWINGS

FIG. 1 is a schematic cross-sectional view of an embodiment of a vacuum assisted liner system for use with a prosthetic device to be attached to a residual limb in accordance with the present invention.

FIG. 2 is a schematic side and cross-sectional view of an embodiment of the vacuum assisted liner system used with a prosthetic device to be attached to a residual limb in accordance with the present invention.

FIG. 3 is a schematic side and cross-sectional view of another embodiment of the vacuum assisted liner system used with a prosthetic device to be attached to a residual limb in accordance with the present invention.

FIG. 4 is a schematic side and cross-sectional view of another embodiment of the vacuum assisted liner system used with a prosthetic device to be attached to a residual limb in accordance with the present invention.

FIG. 5 is a schematic side and cross-sectional view of another embodiment of the vacuum assisted liner system used with a prosthetic device to be attached to a residual limb in accordance with the present invention.

FIG. 6 is a schematic side and cross-sectional view of another embodiment of the vacuum assisted liner system used with a prosthetic device to be attached to a residual limb in accordance with the present invention.

FIGS. 7A and 7B are a schematic cross-sectional views of embodiments of a vacuum assisted liner system including a suspension pin in accordance with the present invention.

FIGS. 8A and 8B are a schematic cross-sectional views of embodiments of a vacuum assisted liner system in accordance with the present invention.

FIG. 9 is a schematic cross-sectional view of another embodiment of a vacuum assisted liner system in accordance with the present invention.

FIGS. 10 and 11 are schematic cross-sectional views of an embodiment of a vacuum assisted liner system including a sealing apron in accordance with the present invention.

FIGS. 12A, 12B and 12C are schematic cross-sectional views of embodiments of a vacuum assisted liner including a sealing apron in accordance with the present invention.

FIGS. 13A and 13B are schematic side views of embodiments of a vacuum assisted liner system including a sealing apron in accordance with the present invention.

FIG. 14 is a schematic cross-sectional view of another embodiment of a vacuum assisted liner system including a sealing apron in accordance with the present invention.

FIGS. 15A and 15B are schematic cross-sectional views of embodiments of a vacuum assisted liner system including a sealing apron in accordance with the present invention.

FIG. 16 is a schematic cross-sectional view of an embodiment of a hypobaric prosthetic liner including a differential pressure mechanism in accordance with the present invention.

FIGS. 17A and 17B are schematic cross-sectional views of embodiments of a hypobaric prosthetic liner including a sealing apron and differential pressure mechanism in accordance with the present invention.

FIG. 18 is a schematic cross-sectional view of another embodiment of a hypobaric prosthetic liner including a sealing apron, differential pressure mechanism, and vacuum relief valve in accordance with the present invention.

FIG. 19 is a schematic cross-sectional view of an embodiment of a vacuum assisted liner system including a differential pressure mechanism and prosthetic sleeve in accordance with the present invention.

FIGS. 20, 21 and 22 are schematic cross-sectional views of embodiments of a vacuum assisted liner system including a prosthetic sleeve in accordance with the present invention.

FIG. 23 is a side and top view of a threaded umbrella connector for use with the vacuum assisted liner system in accordance with the present invention.

FIG. 24 is a side view of an embodiment of a vacuum pump for use with the vacuum assisted liner system in accordance with the present invention.

FIG. 25 is a schematic diagram of an embodiment of a remote control actuated vacuum relief valve system for use with the vacuum assisted liner system in accordance with the present invention.

FIG. 26 is a schematic diagram of an embodiment of a socket and insert for use with the vacuum assisted liner system in accordance with the present invention.

FIG. 27 is a side view of an embodiment of a post operative prosthesis for use with the vacuum assisted liner system in accordance with the present invention.

FIGS. 28A-28C are schematic cross-sectional views of embodiments of a vacuum assisted liner system in accordance with the present invention.

FIG. 29 is a schematic diagram of an embodiment of a “seal-in” liner for use with the vacuum assisted liner system in accordance with the present invention.

FIG. 30 is a schematic cross-sectional view of an embodiment of a vacuum assisted liner system including a sealing membrane in accordance with the present invention.

FIG. 31 is a schematic cross-sectional view of an embodiment of a vacuum assisted liner system including a sealing hose barb in accordance with the present invention.

FIG. 32 is a schematic cross-sectional view of an embodiment of a vacuum assisted liner system including a vacuum gauge in accordance with the present invention.

FIG. 33 is a schematic cross-sectional view of an embodiment of a vacuum assisted liner system including a vacuum relief valve and wedge shaped gasket in accordance with the present invention.

FIG. 34 is a schematic cross-sectional view of an embodiment of a vacuum assisted liner system including proximal vacuum ports in accordance with the present invention.

FIG. 35 is a schematic cross-sectional view of an embodiment of a vacuum assisted liner system including a distal vacuum port in accordance with the present invention.

FIGS. 36A and 36B are schematic cross-sectional views of embodiment of a vacuum assisted liner system including a hose barb and flexible tubing in accordance with the present invention.

FIGS. 37-41 are schematic cross-sectional views of embodiments of a vacuum assisted liner system including a flexible tubing in accordance with the present invention.

FIGS. 42A and 42B are schematic cross-sectional views of an embodiment of a liner system including a sealing apron in accordance with the present invention.

FIGS. 43A and 43B are schematic cross-sectional views of embodiments of a vacuum assisted liner system including a hollow locking pin in accordance with the present invention.

FIG. 44 is a schematic cross-sectional view of an embodiment of a prosthetic wicking sock for use with a vacuum assisted liner system in accordance with the present invention.

FIG. 45 is a schematic cross-sectional view of an embodiment of a hypobaric liner including a tapered passageway for use with a vacuum assisted liner system including in accordance with the present invention.

### DETAILED DESCRIOTION OF THE PREFERRED EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

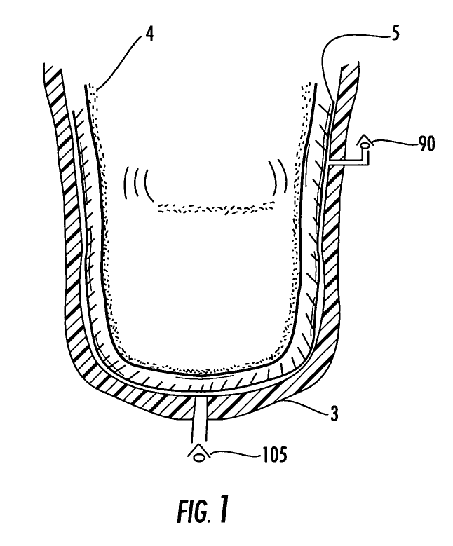

Referring initially to FIG. 1, a hypobaric prosthetic liner 3 which is a component of the Vacuum Assisted Heat/Perspiration Removal System and Limb Volume Management for Prosthetic Device design, will be described. A thin prosthetic sock or wicking material 5 is worn between the residual limb 4 and hypobaric prosthetic liner 3. Air logic symbols at the inlet 90 and outlet 105 show a directional restriction of air flow into and out of the liner, respectively. A vacuum source is ultimately connected at the checked outlet 105, drawing the residual limb 4 and liner 3 into tight adherence via the wicking and communication function of the prosthetic sock 5. The regulation of vacuum occurs both at the vacuum source (of various embodiments found in this application) and via the checked inlet 90 (also of various embodiments), which acts a vacuum relief and regulating valve. Atmospheric air is drawn into the liner 3 at the checked inlet 90, dissipating heat and evaporating perspiration while maintaining a constant regulated vacuum between the residual limb 4 and liner 3. Air, heat, vapor and perspiration are removed by the vacuum source via the checked outlet 105.

Vacuum is negative pressure (or less than atmospheric pressure) commonly expressed in inches of mercury (″Hg) or millimeters of mercury (mmHg) which is equal to torr. One atmosphere equals 14.7 psia (0 psig), 29.92″Hg (0″Hg absolute), 760 mmHg, 760 torr or 1,013 mbar.

The hypobaric prosthetic liner 3 is to be donned over the residual limb 4, and the porous wicking material layer 5 surrounds at least a portion of the residual limb and defines a regulated vacuum environment between the hypobaric prosthetic liner and the residual limb. Passageways are defined by the vacuum inlet and outlet ports which are in fluid communication with the regulated vacuum environment.

It should be noted that the wicking action of the thin prosthetic sock 5, which allows air to flow through it, is the mechanism by which the air inlet 90 and outlet 105 are in communication with each other and allows inflowing air and maintained vacuum to cool the residual limb 4. Although a below knee residual limb is represented in FIG. 1, this configuration can be used on above knee, below knee and upper extremity amputees, as would be appreciated by those skilled in the art.

When air is removed from inside the liner 3, atmospheric pressure pushes the skin of the residual limb 4 and the inside of the liner 3 into tight adherence. This results in the transferring of load and shear and minimizing pistoning of the liner relative to the residual limb 4. It should be pointed out that there is no increase in radial compression of the liner upon the residual limb because of the application of regulated vacuum. There is, however, a dramatic increase in intimate contact between the liner and the residual limb. It should also be noted that the application of regulated vacuum to the inside of the liner does not engender edema in an ideally working system. Again, vacuum draws the liner and the skin into tight adherence.

Creating an elevated vacuum environment between the limb 4 and liner 3 will evaporate the most energetic molecules of perspiration on the residual limb. Adding a mass air flow mechanism accomplished by the vacuum source and the inlet 90 evaporates the perspiration with increased rapidity because of the inflow of air. Elevated vacuum, by lowering saturation vapor pressure, increases the tendency of water to overcome its surface tension and evaporate, which is an endothermic (net heat loss) reaction. The system represented by FIG. 1 includes both elevated vacuum and mass air flow being pulled through the system. The more air pulled though the liner, the more energy removed per unit time.

Referring to FIG. 1, limb volume management is achieved in this design. During the swing phase of the amputee's gait, the weight of the artificial limb, which is suspended on the prosthetic liner 3 (in FIG. 1, the socket is not pictured and suspension is of various embodiments found in this application) and angular acceleration pull on the liner 3. The liner 3, which is in tight adherence with residual limb 4 because of the application of vacuum, causes traction of the distal tissues relative to the tibia, which increases the average volume of distal tissue, creating negative pressure inside the limb, resulting in interstitial refill. Vacuum applied to a prosthetic liner 3 is theorized to improve circulation and increase the oxygenated blood flow to the residual limb.

It should be noted that the prosthetic sock 5 can be replaced by a design encompassing an integrated breathable fabric or membrane attached or bonded to the inside of the liner 3. A bonded breathable fabric serves the same function as the removable prosthetic sock 5, distributing vacuum, removing perspiration and allowing communication between the checked inlet 90 and the checked outlet 105. It may however be desirable to have the ability to remove the prosthetic sock 5 from the liner for hygienic purposes as it also serves as a filter for the pump system capturing dirt particles and mineral deposits. It should also be pointed out that the liner 3 can be manufactured using urethane, silicone, thermoplastic elastomer, RTV rubber or any appropriate material.

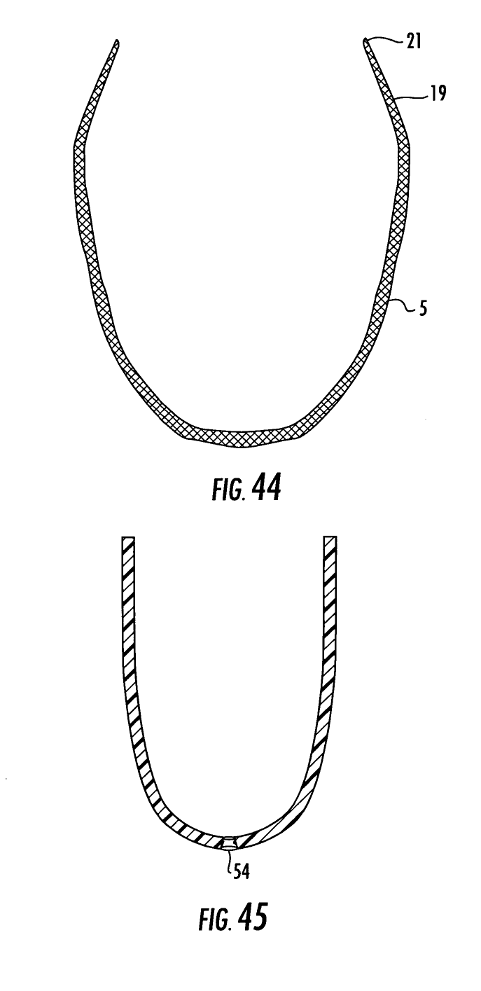

The vacuum level established inside the hypobaric prosthetic liner 3 may be tailored to each patient. The minimal amount of negative pressure to hold the liner 3 on is a function of the weight of the prosthesis divided by the cross-sectional area of the residual limb 4 near the distal end. For example, a below knee patient might require −0.736 PSI to hold their liner and leg on or 1.5″Hg vacuum. A vacuum level of 3″Hg vacuum would be chosen as the vacuum level inside the hypobaric prosthetic liner 3 to achieve a safety factor of 1 (unity). In an average above knee (AK) limb, a zero safety factor would be 3″Hg. Given that most of the problems with elevated vacuum in an artificial limb occur around the popliteal fold (back of the knee), an AK limb on a healthy patient would be able to tolerate 6″Hg if the inside sock were tapered proximally with a band of thin silicone, for example, as detailed in the prosthetic sock 5 shown in FIG. 44.

The chosen vacuum level between the limb and hypobaric prosthetic liner is preferably a clinical judgment of the attending prosthetic practitioner. For example, diabetic patients have thin glassy skin subject to breakdowns. Given the unquantifiable aspect of radial compression and frictional adhesion holding the liner on the residual limb, the safety factor of maintained vacuum between the limb and liner could be lowered in view of protecting delicate tissue. The higher the maintained vacuum level inside the liner, the greater the detrimental effect of problems resulting from the system not functioning perfectly. An example of non-optimal functioning of the system would be wrinkles caused by bending the knee, unintentional gaps or holes in the sock 5, unnoticed curling of the proximal edge of the sock. Each of these occurrences can damage the skin at higher vacuum levels. Accordingly, it is preferable, based upon clinical judgment, that the vacuum level should not exceed 10″Hg between the limb and the hypobaric prosthetic liner. In general, the vacuum level above the minimal amount to hold the limb on, as represented by a safety factor of 1 (or double the minimal amount) improves adherence of fit, evaporation of perspiration, maintenance of daily volume, immobilizes the skin, which reduces the relative motion and transfers load and shear to the liner.

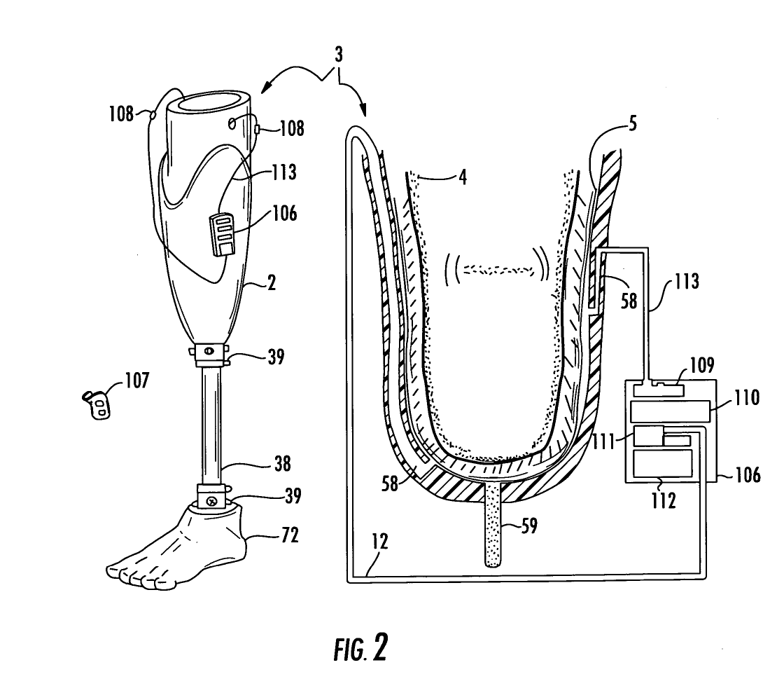

Referring now to FIG. 2, an embodiment will be described of the Vacuum Assisted Heat/Perspiration Removal System and Limb Volume Management for Prosthetic Device outfitted with radio controlled electric vacuum pump 111 and solenoid valve 109, encased in a housing or electronics enclosure 106, as the mechanism of creating and regulating air inflow and vacuum level between the residual limb 4 and the Hypobaric Prosthetic Liner 3. The prosthetic sock 5 functions as described in FIG. 1 in that the wicking action allows air to flow through it, and is the mechanism by which the solenoid valve 109 and vacuum pump 111 are in communication with each other. The thin prosthetic sock 5 facilitates the inflowing air and maintained vacuum to cool the residual limb 4 and remove perspiration. The vacuum pump 111 maintains a preset vacuum level in the closed system of residual limb 4, sock 5 and liner 3 via solid state or microprocessor controls found on an ultra low power RC receiver, encoder, and driver and function circuit board 110 found in the small enclosure 106.

When the appropriate keychain radio transmitter 107 button is pushed, the solenoid valve changes state and opens, allowing air to in flow between the residual limb 4, sock 5, and liner 3. Simultaneously, the vacuum pump 111 controlled, for example, by a pulse width modulation chip on the circuit board 110, maintains a constant RPM effectively pulling air though the system that includes the residual limb 4, sock 5 and liner 3. Technically, air is not actually pulled through the defined system; it is pushed by atmospheric pressure. When the appropriate keychain radio transmitter 107 button is pushed, the solenoid valve changes state and closes, and the system is closed and the maintained and regulated vacuum level is restored.

This configuration can be used on above-knee, below-knee and upper-extremity amputees. The below knee artificial limb illustrated in FIG. 2 is has a prosthetic foot 72, tube clamp adaptors 39, pylon 38, socket 2, external vacuum passage way from the pump 12, electronics enclosure 106, proximal tubing from the solenoid valve 113, quick detachable tube locking mechanism and fine filter 108 and prosthetic liner 3. Internal vacuum passageways 58 in the liner 3 are extensions of external tubing from the vacuum pump's enclosure 106 and solenoid valve 109. The solenoid valve line 113 enters the side of liner and has a shorter internal hose that places the opening or vacuum port just below the politeal fold level and in communication with the prosthetic sock 5. The vacuum pump line exits out the proximal edge of the liner 3 and has an vacuum port in the distal aspect of the liner. The passageways allow air to enter between the limb 4 and liner 3 (in fluid communication with the prosthetic sock 5) as well as remove air via the vacuum pump 111.

A rechargeable lithium battery 112, for example, is the power source for the electronics contained in the enclosure 106. A car cigarette lighter adaptor may also be included with the design to recharge the batteries while traveling. The electric vacuum pump 111, associated controller 110, flow control switch or solenoid valve 109, battery 112, housing 106 and remote control transmitter 107 may define a vacuum regulation device.

The illustrated hose lines 113, 12 may use 0.0625″ ID or 0.040 ID flexible tubing. The smaller ID tubing minimizes noise of the vacuum pump as well as limits the amount of dead air space needed to be removed from the system, making the operation of the vacuum pump more efficient. Internal vacuum passageways 58 may be made from the same tubing cast in the liner 3.

Illustrated in FIG. 2 is a pin lock 59 with internal vacuum passageways 58 cast in the liner 3 walls. The pin lock system was chosen for ease of illustration. There are many different mechanisms of suspension that can be used with the hypobaric Prosthetic Liner which is a fundamental component of the Vacuum Assisted Heat/Perspiration Removal System and Limb Volume Management for Prosthetic Device. These different suspension mechanisms and the alternate embodiments of the Hypobaric Prosthetic Liner will be detailed in various embodiments described below.

It is entirely possible that higher levels of automation may be required to address individual patient needs. A closed system moisture/humidity sensor may augment or replace the keychain remote function of initiating the boost flow of cooling air through the typically closed limb 4, sock 5, and liner 3 system. An ambient air humidity sensor maybe added to automatically adjust the vacuum level inside the limb 4, sock 5, and liner 3 system. A temperature sensor could also be used for this function, as the temperature inside the liner (relative to ambient air) rises, the pump could go through a vacuum/air flow boost cycle, rapidly cooling the limb. An accelerometer could also be used so that as relative motion (i.e. an increase in activity) increases, the vacuum level could be raised and cooling take place. A vacuum sensor actuating a solenoid valve may also be employed, defining a vacuum regulation device.

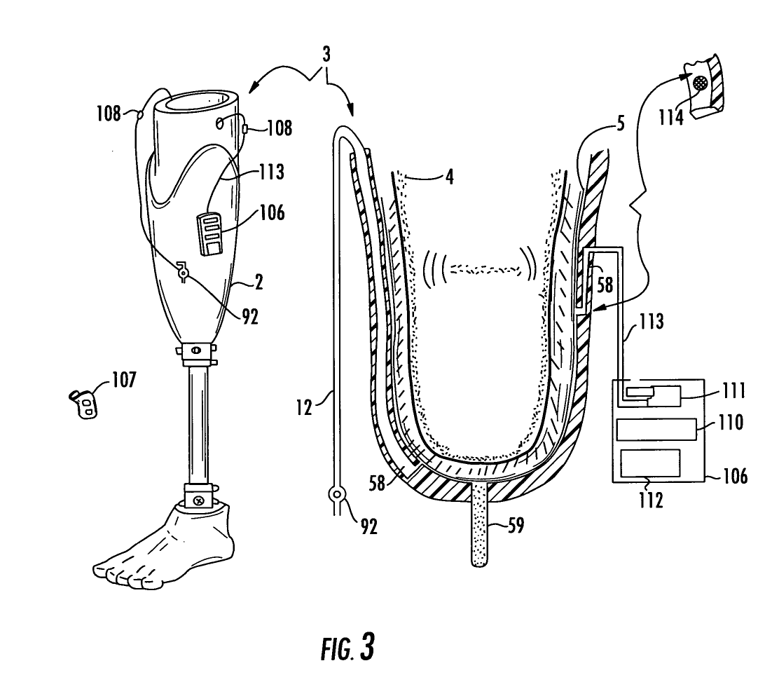

Depicted in FIG. 3 is an alternate embodiment of the Vacuum Assisted Heat/Perspiration Removal System and Limb Volume Management for Prosthetic Device outfitted with a radio controlled electric vacuum pump 111. This embodiment dispenses with the electronically controlled solenoid valve. This design uses a manually adjustable vacuum relief valve 92 that is attached to the prosthetic socket 2 and is connected to vacuum relief line 12. In this configuration the vacuum pump 111 is connected relatively proximal on the limb 4 and the relief valve mechanism is ported distally though internal tubing 58 cast in the liner 3. Any relative placement of the relief valve and vacuum pump is possible. The manually adjustable vacuum relief valve 92 is sensitive and repeatable, accurate to a narrow band of vacuum. It is, therefore, ideally suited with the use of a battery 112 powered vacuum pump 111 contained in the enclosure 106 that defines the vacuum pump system. The enclosure 106 is attached via the proximal tube 113 to the liner 3 and internal vacuum passageway 58.

There is an optional mesh filter 114 that covers the entry point for the proximal tube 113, and although not illustrated, there may be other optional mesh filters that cover, for example, the distal entry point of vacuum on the inside of the liner 3. The mesh filters 114 act as a crude filter for the vacuum pump and prevent potential window edema given the thin ply prosthetic sock 5. A quick detachable tube locking mechanism and fine filter 108 may be located in the external tube lines 12, 113. The electric vacuum pump system maintains an adjustable and regulated vacuum inside the prosthetic liner 3 with solid state or microprocessor controls found on the ultra low power, radio control receiver, encoder and driver and function circuit board 110 located in the enclosure 106. This configuration can be used on above-knee, below-knee and upper-extremity amputees.

When the appropriate button on the keychain radio transmitter 107 is depressed, the radio control receiver 110 initiates a signal that increases the RPM of the motor, raising the vacuum level, activating the vacuum relief valve that allows air to flow through the system, rapidly cooling the stump. Air enters the system in FIG. 3, though the manually adjusted vacuum relief valve 92, which is connected to the vacuum relief tubing 12 and in communication with the internal vacuum passageway 58, and the prosthetic sock 5, which allows air out the second internal vacuum passageway and out via the pump 111 and ultimately expelled to the atmosphere. The boost feature is ceased when the user presses the correct off button. Alternate designs may incorporate a timed feature, so that when one button is pushed the pump runs a high RPM for a preset time. More automated features may be incorporated into the design as discussed above in FIG. 2.

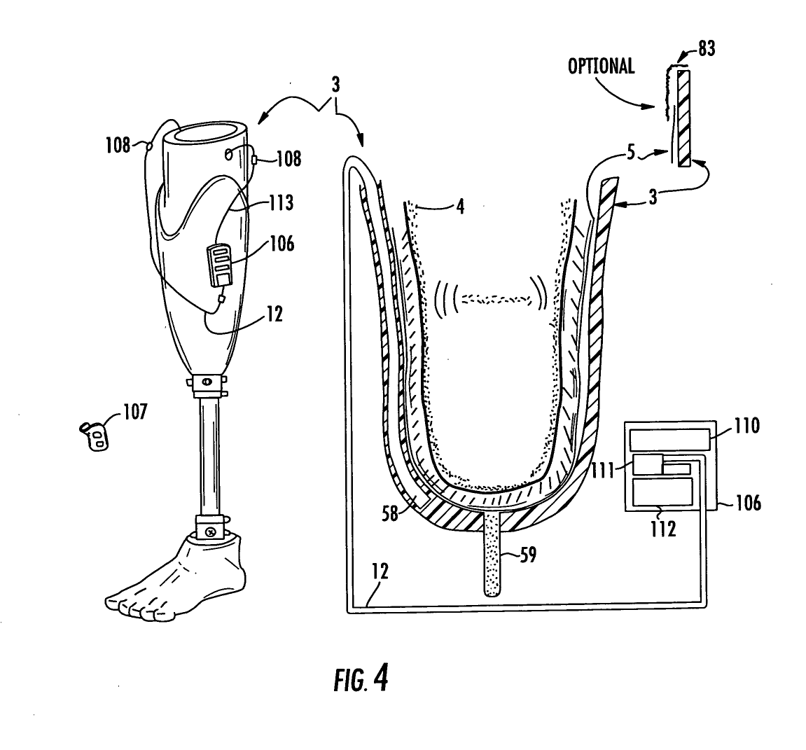

Depicted in FIG. 4 is an alternate embodiment of the Vacuum Assisted Heat/Perspiration Removal System and Limb Volume Management for Prosthetic Device. This embodiment employs only regulated vacuum to the system that includes the limb 4, sock 5, and liner 3. A regulated but elevated vacuum will cool the limb by facilitating evaporation (lowering the vapor pressure) which is a net heat loss event. Regulated vacuum is supplied via a battery 112 powered vacuum pump 111 controlled by either a digital or analog vacuum sensor found on the circuit board 110. The vacuum pump 111 is connected to the distal tubing, or vacuum passageway 12, which is in communication with the internal vacuum passageway 58 and also in communication with the thin prosthetic sock 5, which acts as a wick and distributes regulated vacuum to the amputee's limb 4. The sock 5 as illustrated is a removable thin ply prosthetic sock. It should be noted again that the design of an integrated breathable fabric or membrane bonded to the inside of the liner 3 would perform the same function. Vacuum supplied to a prosthetic limb with this configuration achieves volume management, perspiration removal, cooling of the stump and minimizing of pistoning. Also achieved is the benefit of the skin being immobilized, thus loads and shear being transferred to the liner.

An optional feature to this embodiment allows atmospheric air to be introduced into the liner by the user via a wick 83. The wick can be a cut piece of prosthetic sock, a section of yarn or any breathable material that will allow air to enter the system that comprises the limb 4, sock 5, and inside hypobaric prosthetic liner 3. The wick 83 can be tucked away in the liner, allowing a homeostatic elevated but regulated vacuum to exist in the closed system. When the wick is extended by the user, so that it protrudes beyond the proximal border of the liner 3, and in communication with the outside air, the vacuum pump 111, turns on and a rapid inflow of air occurs into the now open system. Control of the surface area of the wick 83, in regards to the mass capacity of the pump, will also maintain an elevated vacuum in the system. When sufficient cooling has been achieved, the user tucks the wick back into the liner and the system is closed and a maintained vacuum level is restored. This described configuration can be used on above-knee, below-knee and upper-extremity amputees.

Although the use of elevated vacuum applied to the outside of a prosthetic liner and the socket is known, the present invention includes the application of vacuum to the inside, or a combination of both inside and outside, as detailed in forthcoming embodiments. The Vacuum Assisted Heat/Perspiration Removal System and Limb Volume Management for Prosthetic Device is not limited to a pin suspension. Although not illustrated in the various embodiments, it should be noted that between the outside of the liner 3 and the socket 2, a nylon, or sock can be worn for comfort and for ease of inserting the limb and liner into the socket.

Depicted in FIG. 5 is an alternate embodiment of the Vacuum Assisted Heat/Perspiration Removal System and Limb Volume Management for Prosthetic Device that uses a motion activated pump or air cylinder 125, configured to create vacuum or sub-atmospheric pressure during each step the user takes. As with all the previous embodiments, regulated vacuum is being delivered to the inside of the prosthetic liner 3, to cool the limb, evaporate moisture and minimize volume changes in the limb during daily wearing.

For example, such a motion activated pump can be found in U.S Pat. Application Pub. No. 2005/0143838 to Collier described as VACUUM-ASSISTED PROSTHETIC DEVICE. Careful reading of the patent reveals that Collier's method draws elevated negative pressure to the inside of the prosthetic socket and on the outside of the prosthetic liner. Collier's system is designed to be used with a specific foot

The embodiment or the present invention draws elevated negative pressure to the inside of the prosthetic liner 3 between the limb, sock, and liner system as described previously. The embodiment presented here is also modular and designed to fit on various limbs, sockets and feet available in the marketplace. The base plate 120 that the air cylinder configured for vacuum generation is bolted to has, for example, four oblong holes 122 (two of the holes are not shown) so the relative placement of the air cylinder configured to create sub-atmospheric pressure can be adjusted, increasing and decreasing the lever arm from the axis of rotation, resulting in increased pull on the piston rod 11). This adjustability prevents off axis loading when different prosthetic feet 72 are employed and adapts to the limits of plantar flexion and dorsiflexion of the various prosthetic feet used. For example, the longer the lever arm is from the center of rotation, the less dorsiflexion and plantar flexion needed to achieve full stroke length of the piston in the air cylinder configured to create vacuum. Attached to the heel of the prosthetic foot 72 with low profile hook and loop with high shear pressure sensitive adhesive is 0.5″ Dacron webbing and a cable hanger 115. Attachment of the Dacron webbing and cable hanger 115 to the shoe is another possibility that would in effect increase the lever arm further from the axis of rotation, resulting in increased pull on the piston rod 119. Stainless steel aircraft cable 116 is connected to a ball receiver that is connected to a ball terminal 118, which is in turn connected to the rotating piston rod 119 in the air cylinder configured for vacuum generation 125.

The air cylinder is configured for generating negative pressure by employing low cracking pressure check valves. The air logic diagrams of check inflow and checked outflow are on either end of the illustrated tee 124. The air cylinder is outfitted with four tie rods 123. The advantage of the tie rod design is that the overall height of the cylinder can be customized to each individual prosthetic foot which is another modular feature. A clamping screw 121 allows the device to be adjustable up and down the pylon 38. A flexible cable 116 connects the foot 72 and the piston rod 119 of the air cylinder configured to generate vacuum. If a rigid linkage was employed instead of the flexible cable 116, vacuum could be generated in both the down pull on the piston rod 119 and the return stroke. Outfitted with a spring return, vacuum is generated only on the pull stroke.

A tiny, lightweight, spring loaded vacuum relief valve is attached to the proximal aspect of the liner 126. This non-adjustable vacuum relief valve 90 is employed to allow air flow inside the prosthetic liner while maintaining a therapeutic vacuum level inside the liner. The use of a non-adjustable vacuum relief valve 90 is best suited for a body powered vacuum pump 125 as the mechanism is not sufficiently accurate enough or exactly repeatable in its vacuum cracking pressure to work well with a battery powered vacuum pump with electronic controls. It should be noted that the non-adjustable vacuum relief valve 126 is well within tolerances for use with the illustrated air cylinder configured for vacuum use 125.

Vacuum is generated with each step; just before foot flat and toe off in the human gait cycle, the shaft 119 of the air cylinder is pulled downward, and air is drawn in from the spring loaded vacuum relief valve 126 which is in communication with the prosthetic sock 5. The prosthetic sock 5 distributes vacuum over the limb and when a set vacuum level is achieved, the vacuum relief valve opens and air is pulled through the liner 3, rapidly evaporating moisture and cooling the limb 4. By the function of the relief valve, a vacuum level is maintained inside the liner 3 as air is constantly streaming in. Air is drawn in between the limb 4 and liner 3 flowing out through the internal vacuum passageway in the liner, out through the external vacuum tubing 12 and into the air cylinder configured as a vacuum pump 125. After toe off and immediately after heel strike, the spring return piston retracts completely eliminating any dead air space, which is expelled via the checked outflow mechanism connected to the tee 124 and the cycle begins again.

The importance of vacuum inside the liner 3 is that it reduces relative motion between the limb and liner, it wicks away moisture and keeps the limb cool. This is important to amputees with vascular complications (e.g. stemming from diabetes) because temperature regulation is a function of circulation cooling the limb, which in such cases is compromised by disease. Heat, perspiration and pressure are leading causes of tissue maceration and wound healing is impeded by dyvascular complications. There is strong research evidence of the efficacy of vacuum assisting in wound healing. The application of vacuum inside the prosthetic liner draws the liner and the skin into tight adherence. There is no compression of the stump nor is there any tendency for edema. The pressure loading of the skin is transferred to the liner because relative motion has been eliminated by the addition of regulated vacuum between the stump and liner. This configuration can be used on above-knee, below-knee and upper-extremity amputees.

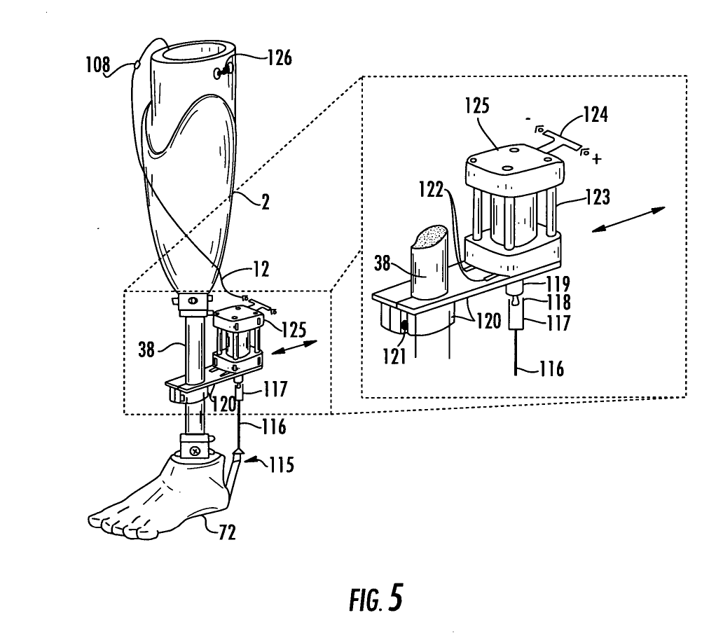

Depicted in FIG. 6 is an alternate embodiment of the Vacuum Assisted Heat/Perspiration Removal System and Limb Volume Management for Prosthetic Device. This embodiment uses only regulated vacuum to the system that includes the limb 4, sock 5, and liner 3. A regulated but elevated vacuum will cool the stump by facilitating evaporation (lowering the vapor pressure) which is a net heat loss event. The air cylinder 125 configured to generate sub-atmospheric pressure supplies regulated vacuum. The vacuum is regulated via a needle valve 127 connected on the air cylinder configured as a vacuum pump. There is a check valve between the needle valve 127 and the liner 3. The vacuum tubing 12 connects to the socket 2 and ultimately inside the hypobaric prosthetic liner 3 via the internal vacuum passageway 58.

The vacuum configured air cylinder is connected to the distal tubing, or vacuum passageway 12, which is in communication with the internal vacuum passageway 58, and also in communication with the thin prosthetic sock 5, which acts as a wick and distributes regulated vacuum to the amputee's limb 4. Vacuum supplied to a prosthetic limb with this configuration achieves volume management, perspiration removal, cooling of the stump and minimizing of pistoning. Also achieved is the benefit of the skin being immobilized, thus loads and shear being transferred to the liner. The optional feature of allowing atmospheric air to be introduced into the liner by the user via a wick 83 as described above, can also be used herewith.

The knitted prosthetic sock 5 described in this application is not an osmotic membrane, it assists in removing heat and perspiration by allowing distribution of vacuum over the residual limb and communicating between the checked inlet 90 and checked outlet 105 mechanism schematically represented in FIG. 1. As described above, the thin prosthetic sock 5 makes possible the evaporation of water vapor because of the maintained elevated vacuum in the prosthetic limb and the wicking action of air being drawn into the liner by the check inlet 90 or vacuum relief/regulating valve.

The design schematically represented in FIG. 1 takes into account the mechanism of applying regulated vacuum directly between the limb 4 and liner 3 assisted by either a sock 5 or integrated breathable fabric in a safe and hygienic fashion, removing heat, moisture, maintaining limb volume. The only way that any perspiration is removed in traditionally outfitted elevated vacuum limbs is at the proximal border of liner, which is sealed by a suspension sleeve. The practice of using a nylon sheath (that is worn between the socket and liner) reflected and tucked inside the liner to wick perspiration has been a field adaptation of the end user, using currently available prosthetic vacuum technology. The nylon does not extend longer then 1.5″ inside the proximal band of the prosthetic liner. The efficiency of such a set up in removing perspiration is limited because the surface covering the limb by the nylon is limited.

It should be noted that designs presented here preferably do not have any prosthetic wicking sock closer then 2″ from the proximal border of the liner. This is done to maintain a redundant vacuum seal on the limb as well as the understanding that the distal areas of the residual limb is where problematic perspiration builds up. It should also be noted that all the vacuum pump designs presented in this application only deliver sub-atmospheric pressure to the inside of the prosthetic liner. In forthcoming embodiments, the Hypobaric Prosthetic Liner will be shown being adapted to designs that have elevated vacuum to the outside of the prosthetic liner (not the inside). Some of the embodiments presented employ vacuum pumps that are commercially available.

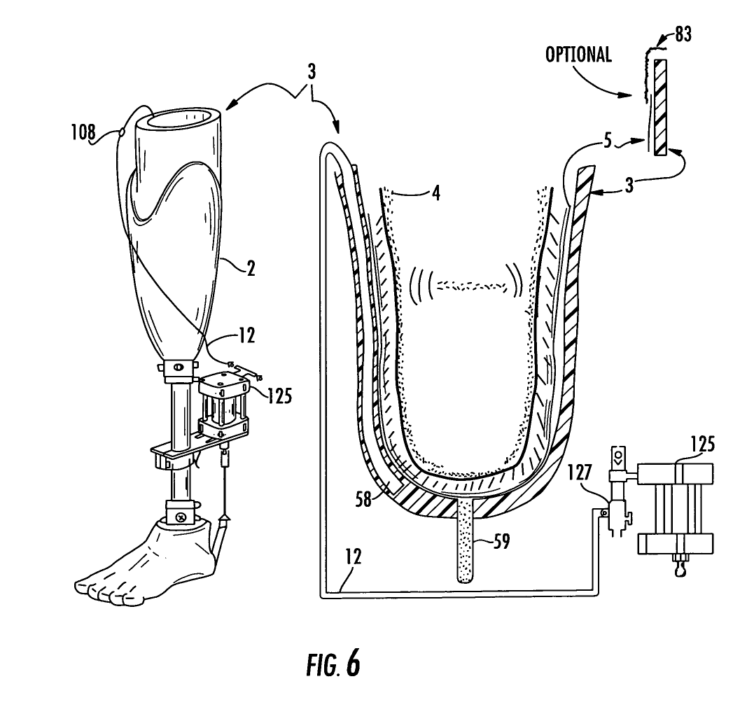

FIGS. 7A and 7B illustrate the basic components of a suspension pin 59 liner 3 with vacuum passageways 58 cast in the walls of the liner. FIG. 7A has one line of vacuum 12 and one internal vacuum passageway 58. Figure B has two lines of vacuum 12, 113 and two internal vacuum passageways 58. The external vacuum tube with the proximal entrance 12 into the liner 3 follows an internal vacuum passageway 58 to a distal vacuum port in the liner 3. The external vacuum tube 113 that enters the wall of the liner 3 that follows an internal vacuum passageway 58 has a relatively proximal vacuum port. As long as the vacuum ports are in communication with the prosthetic sock 5 the relative placement of both ports is variable. It is, however, best to achieve some separation between the two.

The vacuum passageways are made with small ID flexible tubing placed in the Hypobaric Prosthetic Liner mold before filling. Suspension of the Hypobaric Prosthetic Liner 3 is solely a mechanical interface of the locking pin 59 and shuttle lock in the socket (not illustrated). The shuttle lock secures the pin via many different methods and releases the pin with a push button mechanism (not illustrated). The advantage of using a pin suspension is the ease and simplicity of the configuration as well as the increased range of knee motion achieved with the removal of the traditional suspension sleeve or other sealing mechanisms. In pin-lock systems, a problematic phenomenon known as pistoning occurs. Pistoning is when distal distraction of the liner occurs because of the weight of the prosthetic limb, which is borne on the distal locking pin. This can cause discomfort, edema, skin irritation and abrasion. When regulated elevated vacuum is applied to a pin lock prosthetic liner, pistoning is minimized, the skin is immobilized, transferring loads and shear from the skin to the liner.

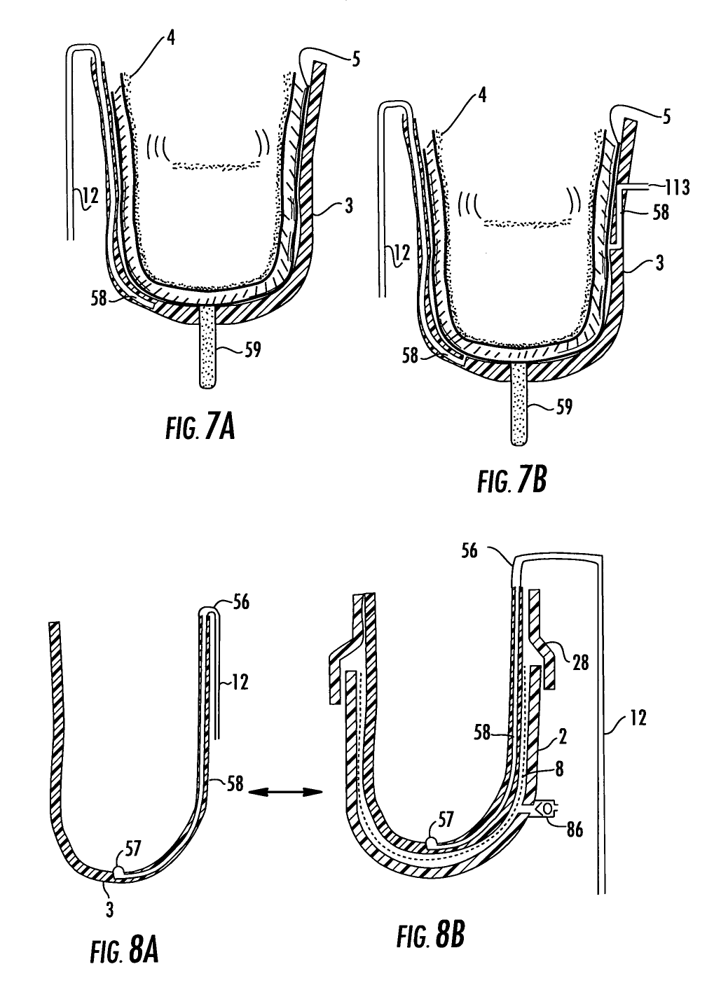

FIG. 8 depicts an alternative embodiment of delivering vacuum to the inside of the Hypobaric Prosthetic Liner 3 and its suspension inside a prosthetic socket 2. An internal vacuum passageway 58 is molded in the wall of the prosthetic liner 3. This could be 0.0625″ ID or 0.040″ ID flexible tubing actually placed inside the mold for the liner and ported, or an actual cavity formed in the wall of the liner. Exiting at the proximal edge of the liner, a proximal external vacuum port 56 allows the use of a prosthetic apron or curtain or sealing sleeve that does not extend above the prosthetic liner 3. A distal internal vacuum port 57 has a tapered conical shape that delivers vacuum to the inside of the prosthetic liner. Although not illustrated, a mesh screen 114 may cover the port 57 as it is illustrated in FIG.2 and FIG. 3.

This embodiment allows the use of a custom prosthetic sleeve 28 and an expulsion valve 86 employed to suspend the limb. The vacuum tubing 12 connects to a vacuum source, either the battery power pump 106, as found in FIG. 4 or the motion activated design 125, found in FIG. 6. This design is not limited to just regulated vacuum being applied to the limb, other embodiments of this design will allow for cooling air inflow into the system. The expulsion valve 86 is a long standing standard practice in prosthetics. It allows air to be expelled, assisted by either a nylon or prosthetic sock 8 on the outside of the prosthetic liner 3 and creates negative pressure to suspend the limb in the liner. Alternate embodiments presented in this application will the show the use of elevated vacuum being applied to the outside of the liner in conjunction with the use of elevated and/or regulated vacuum applied to the inside of the liner 3.

A prosthetic sock (not illustrated here) distributes regulated vacuum to the limb of the amputee. An external vacuum passageway 12 connects to a vacuum source. So long as the distal internal vacuum port 57 interfaces with the vacuum distributing sock, there is no reason why it has to be specifically located at the distal center of the prosthetic liner. It could be located anywhere proximally up the walls of the prosthetic liner 3. It should be pointed out that there is no reason as to why this configuration could not be used with a traditional solid pin lock liner as depicted in FIG. 7. This configuration can be used on above-knee, below-knee and upper-extremity amputees.

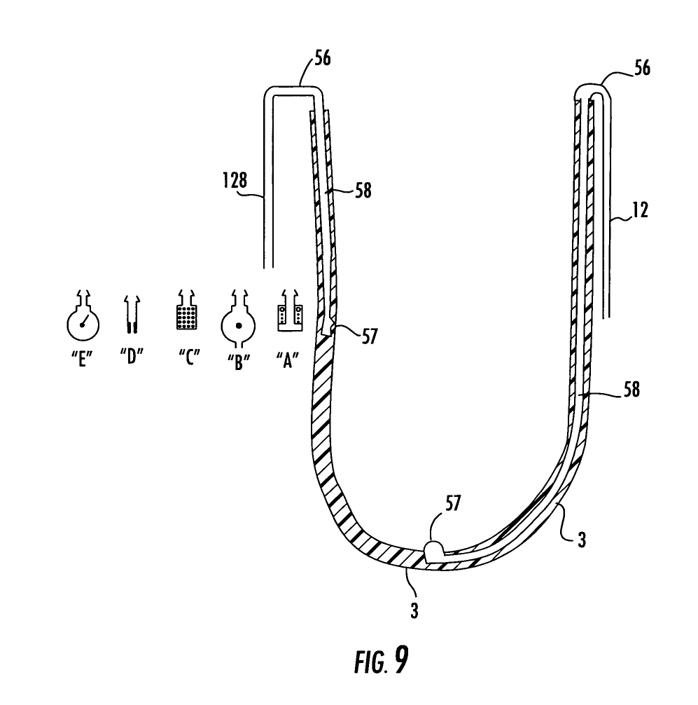

Depicted in FIG. 9 is an alternate embodiment of the Hypobaric Prosthetic Liner 3 that employs two internal vacuum passageways 58 and two internal vacuum ports 57. The two proximal external vacuum ports 56 allow air into and out of the Hypobaric Prosthetic Liner. This configuration can be used with a sealing sleeve, a tapered wedge or even a prosthetic apron liner bonded to the proximal edge of the liner 3, as will be described below, as well as above the trim lines of the socket 2. The vacuum passageway/hose 128 allows a number of fittings A-E to be inserted for different functions. Insert “A” is a non-adjustable vacuum relief valve which would be used if a body powered vacuum pump were attached to the vacuum passageway/hose 12. Insert “B” is an adjustable vacuum relief valve that would be used with a battery powered vacuum pump because of its greater sensitivity and repeatability. Insert “C” is a push button relief valve that would be used create a free flow of air through the prosthetic liner. Insert “D” is a micro drilled orifice that would allow controlled leaking of air into the liner 3, which would necessitate a body powered vacuum pump. Insert “E” is a vacuum gauge that could be attached to give an accurate readout of the vacuum level inside the Hypobaric Prosthetic Liner 3.

Air pulled by the vacuum source attached to passageway/hose 12, travels through the a given insert, up the hose/passageway 128 into the first proximal external vacuum port 56 down the internal passageway and out the internal proximal vacuum port 57 distributed over the limb by the thin prosthetic sock (not pictured) down through the distal internal vacuum port 57 and up the internal vacuum passageway 58 and out the second proximal external vacuum port 56 and down the external passageway/hose 12 towards the vacuum source. This configuration can be used on above-knee, below-knee and upper-extremity amputees.

A custom manufactured suspension sleeve can be manufactured from a patient's custom measurements. Suspension sleeves are in common practice in prosthetic limb designs, and can be basically described as an elastic tube which covers the socket and a part of the amputee's limb. In an elevated vacuum suspension artificial limb design, a suspension sleeve provides redundant suspension, connecting the prosthetic socket and liner via frictional adhesion, as well as a vacuum seal between the limb and the socket. It is important to understand that traditional suspension sleeves, employed in an elevated vacuum or hypobaric sockets, are of standard stock dimensions and not customized to the patient's limb and liner. A suspension sleeve 28, such as illustrated in FIG. 8, can be manufactured from custom measurements that take into account the shape of the Hypobaric Prosthetic Liner 3 and socket 2. Using custom measurements allows a minimal amount of compression to be used in suspending and sealing the prosthetic limb and an exceptional fit is achieved. The suspension sleeve with traditional stock dimensions is difficult to don, can be excessively tight on the amputee's limb and does not maintain an air tight seal.

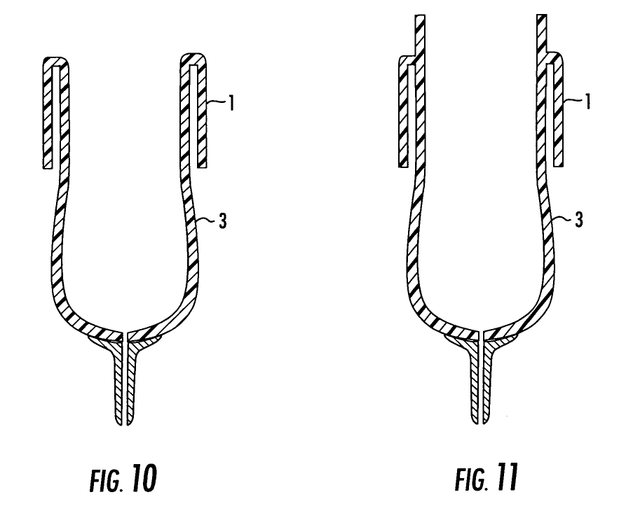

FIGS. 10 and 11 depict alternative embodiments of the Hypobaric Prosthetic Liner 3 used in the Vacuum Assisted Heat/Perspiration Removal System and Limb Volume Management for Prosthetic Device design. A unique proximal sealing and suspension mechanism is used in this embodiment of the Hypobaric Prosthetic Liner 3. Both versions illustrate the different placement of a vacuum sealing and suspension apron or curtain 1 on the liner 3. The FIG. 10 illustration shows a proximal placement of the apron or curtain bonded to the edge of the liner 3. The FIG. 11 illustration shows a placement of the apron or curtain that is above and follows the trim lines of the socket.

The apron or curtain 1 is employed to minimize vacuum leakage on systems that employ elevated vacuum to the outside of the prosthetic liner and it assists in providing positive suspension for the prosthesis. The Apron/Curtain is hermetically bonded or molded or adhered to the prosthetic liner 3 creating a monolithic airtight structure, i.e. integrally formed. The FIG. 10 illustration could also be molded in such a way as to facilitate it being reflected down onto the proximal aspect of the socket creating an air tight seal. It should be noted that the application of a tacky silicone adhesive sticking the liner and apron/curtain together (but not permanently adhered), which will allow the curtain or apron to be removed and reapplied is also a viable way to achieve this embodiment, whose objective is to create an air tight proximal seal between the liner and apron or curtain. This configuration can be used on above-knee, below-knee and upper-extremity amputees. It is used in versions of the Hypobaric Prosthetic Liner that have elevated vacuum applied to both the inside of the liner and the outside of the liner, which will be discussed in further detail below.

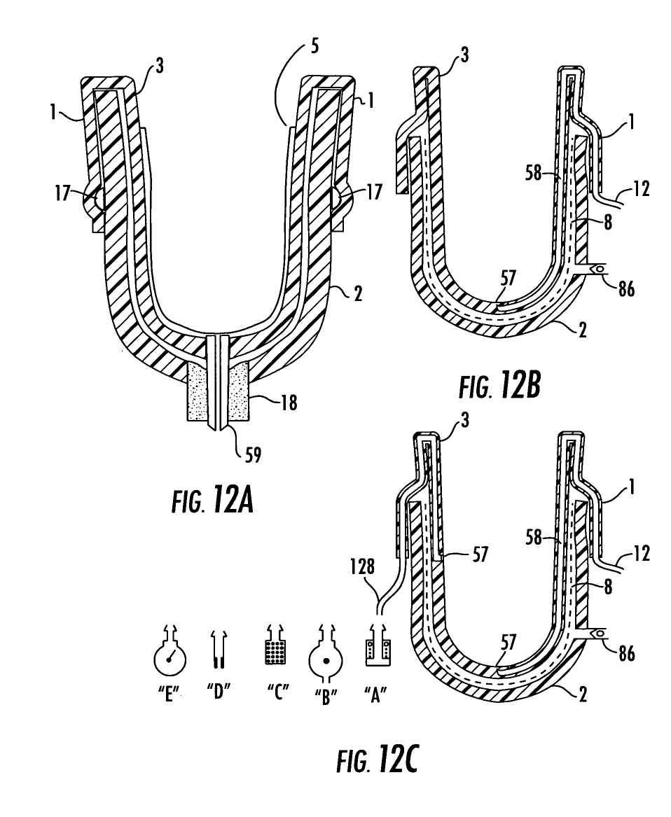

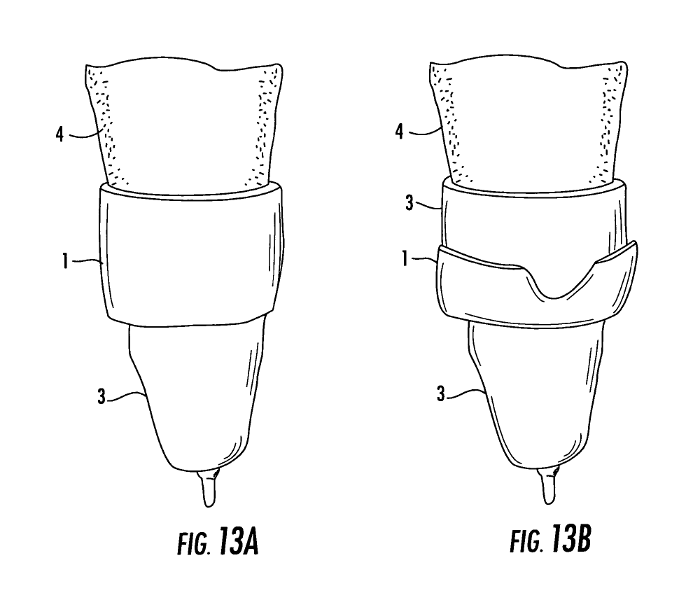

Referring to FIGS. 12A-12C, the apron or curtain 1 is fitted over the prosthetic socket 2 of an artificial limb, providing positive suspension and creating an airtight seal between the liner and the socket. Illustrated in FIGS. 12A-12C are various embodiments of the Hypobaric Prosthetic Liner 3 that use the hermetically bonded apron curtain 1. In the socket and liner design in FIG. 12A an optional ½ round o-ring is bonded circumferentially around the socket and under the apron/curtain 1 of the Hypobaric prosthetic liner 3 to provide added vacuum sealing capacity. Although not illustrated here, this socket 2 could also be redundantly suspended via traditional suction suspension employing a one-way valve through the wall of the socket 2 or elevated vacuum applied to the outside of the liner 3.

The liner and socket design of FIG. 12B is outfitted with an internal vacuum passageway 58 passing though the body of the liner and the apron/curtain 1. It is also outfitted with an internal vacuum port 57. The vacuum tubing 12 connects to a vacuum source, either the battery power pump 106, like found in FIG. 4 or the body powered design 125, found in FIG. 6. The expulsion valve 86 allows air to be expelled, assisted by either a nylon or prosthetic sock 8 on the outside of the prosthetic liner 3 and creates negative pressure to suspend the prosthesis on the liner.

The liner and socket design of FIG. 12C is similar to the embodiment described in FIG. 9, with the difference being the dual internal tubing 58 passing through the hermetically bonded apron/curtain 1. It has a second internal vacuum passage way 58 and a second vacuum port. Optional inserts attach to the external tubing 12, as defined in FIG.9, and are available to customize the liner. The expulsion valve 86 allows air to be expelled, assisted by either a nylon or prosthetic sock 8 on the outside of the prosthetic liner 3 and creates negative pressure to suspend the prosthesis on the liner.

The stretch and elongation of the material employed in the prosthetic liner, be it urethane, silicone, thermoplastic elastomer, or RTV rubber, allows the curtain/apron to be pulled over the prosthetic socket 2 and conform, creating an airtight seal. Again, the apron or curtain can either be permanently bonded or stuck together with a special high bond tacky silicone, allowing the curtain or apron to be removable. The objective of the apron or curtain is to create an air tight seal proximally on the prosthetic socket. If the vacuum seal is ever compromised, the curtain suspends the prosthetic socket via frictional adhesion. This configuration can be used on above-knee, below-knee and upper-extremity amputees.

It is worth noting that a traditional suspension sleeve employed in an elevated vacuum or hypobaric socket has two potential leak areas. One leak path occurs distally where it clings to the socket, and the other occurs proximally on the amputee's skin. Clinical experience indicates that off the shelf, standard sized suspension sleeves are difficult to don, limit knee flexion, are excessively tight on the skin and provide at best, a leaky vacuum seal. The curtain/apron embodiment of the Hypobaric Prosthetic Liner addresses of these problems.

As depicted in FIGS. 13A and 13B, the apron or curtain 1 eliminates one of the vacuum leak paths encountered when using a traditional suspension sleeve in an elevated vacuum suspended limb. The leak path eliminated is where the suspension sleeve interfaces with the amputee's limb 4. Both versions illustrate the different places where a vacuum sealing and suspension apron or curtain 1 is attached to the liner 3. The FIG. 13A illustration shows a proximal placement of the apron or curtain and the FIG. 13B illustration shows a placement that is just above the trim lines of the socket. The apron or curtain 1 is attached in an air tight fashion or molded directly to the liner 3, allowing elevated vacuum only to the inside of the prosthetic socket and the outside of the prosthetic liner 3. This configuration does not permit the amputee's limb 4 to be exposed to unregulated vacuum, which can cause discomfort and skin problems. A pin suspension is illustrated in FIGS. 13A and 13B, the design is not limited to a hollow pin to deliver vacuum to the inside of the prosthetic liner 3. This configuration can be used on above-knee, below-knee and upper-extremity amputees.

Referring to FIG. 13, the outside of the apron/curtain 1 may have a friction reducing covering applied so as not to irritate the contralateral limb as might be experienced in transfemoral applications. A friction reducing covering would also fit inside clothing more easily as in transtibial and UE limbs. The covering (nylon fabric for example) will also protect the curtain/apron 1 from abrasion and allow it to be reflected and slide over itself with out difficulty when donning. A Teflon coating created by vapor depositing is also a possibility. The distal aspect of the liner 3 may also have a reinforcing and friction reducing covering applied to the outside of the liner. For purposes of creating an air tight seal, the underside of the curtain/apron is left uncovered, as well as the proximal two inches of the outside of the liner 3.

Tension or reduction values are a common reference in the manufacture of a prosthetic socket or liner. It refers to the difference between the patient's limb circumferential measurements and the positive model that is used to create a custom prosthetic liner or socket. Although the Hypobaric Prosthetic Liner 3 can be either of custom measurements or standard average sizes, there are advantages to custom molding of the curtain or apron. Using custom measurements allows a minimal amount of compression to be used in suspending and sealing the prosthetic limb. Under 0.5″ PSI of compression caused by the preload of the liner will be the stated objective of the Hypobaric Prosthetic Liner, as research has shown this to be the maximum amount that tissues can tolerate without shrinking.

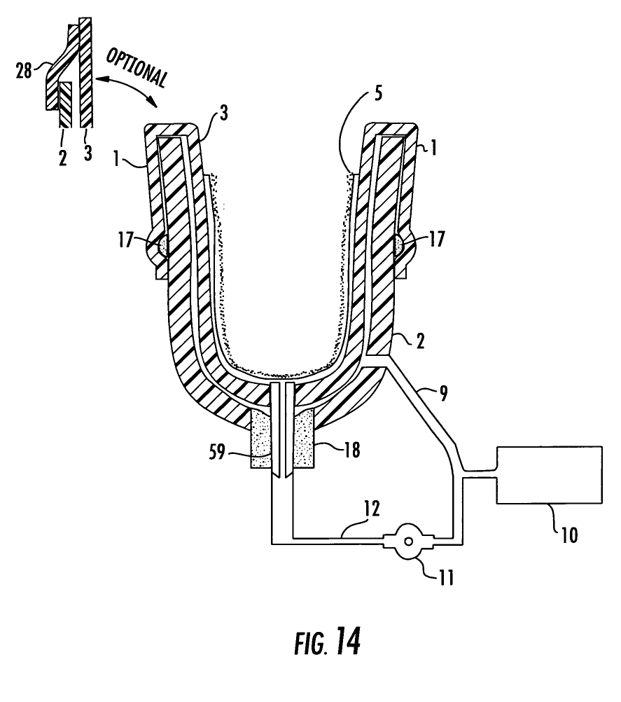

FIG. 14 shows an alternate embodiment of the Hypobaric Prosthetic Liner 3 that employs the proximal apron/curtain embodiment. Using a custom sleeve 28 (or off the shelf) is illustrated as an option. The illustrated Hypobaric Prosthetic Liner 3 has elevated vacuum applied simultaneously to the outside of the liner as well as the inside. Vacuum pumps, configured to be used on artificial limbs between the socket and outside of the liner, are commercially available from manufacturing and supply companies marketing to the prosthetic industry. A vacuum pump 10 increases the differential pressure within the socket 2 of an artificial limb. This increased suction or negative pressure assists in suspending a limb and socket 2 upon an amputee. The Hypobaric Prosthetic Liner 3 employs a unique application of elevated vacuum delivered to the inside of a prosthetic liner or both the inside and outside of the liner in an artificial limb.

A commercially available vacuum pump 10 of any configuration (body powered or battery operated) delivers vacuum to the outside of the prosthetic liner 3 via flexible vacuum tubing 9 connecting to the prosthetic socket 2, which is a standard practice in prosthetics. It is important to note that elevated vacuum applied to the outside of the Hypobaric Prosthetic Liner 3 is achieved by commercially available vacuum pumps. The unique vacuum pump systems presented previously in this application only deliver vacuum only to inside of the liner 3. This design branches off a line of vacuum going to the socket 2 and delivers it to the inside of the liner 3. As illustrated, a small adjustable vacuum regulator 11 delivers regulated elevated vacuum via the flexible vacuum tubing 12 to the hollow locking pin 59 secured in the shuttle lock 18 of the socket 2. This design is not limited to a hollow suspension pin 59 as forthcoming embodiments will illustrate.

Referring to FIG. 14, a vacuum regulator 11, whether mechanical, analog or digital, regulates the vacuum level from the vacuum pump 10 though the passageway 12 to the hollow locking pin 59 of the Hypobaric Prosthetic Liner 3. Employing a mechanical vacuum regulator 11 allows only one sensor to be placed on the high vacuum side (vacuum to the outside of the liner). So long as vacuum is maintained to the outside of the liner, which is higher then the vacuum level to the inside of the liner, the required vacuum levels for both the inside the liner and outside will be achieved by use of a mechanical vacuum regulator 11. The use of a prosthetic sock 5 permits vacuum to be drawn directly on the limb by allowing vacuum to wick through the fibers of the sock allowing an even distribution of differential pressure.

A prosthetic sock 5 designed to be worn between the stump and the liner is employed as a sweat wicking sock in the Hypobaric Prosthetic Liner 3, which allow vacuum to be distributed evenly over the limb 4. Although these socks are designed to be worn between the skin and the liner, the use as a sweat wicking sock, as well as for vacuum distribution, is unique to the Hypobaric Prosthetic Liner of the Vacuum Assisted Heat/Perspiration Removal System and Limb Volume Management for Prosthetic Device design. Regulated vacuum between the liner and the skin, assists in minimizing volume loss by bringing the liner and the skin in tight adherence, which, during swing phase, causes distal traction of the tissues relative to the bones of the stump, and facilitates interstitial refill of the limb. It also assists in removal of perspiration, immobilizes the skin, and transfer load and shear to the liner.

Referring to FIG. 14, it should be pointed out that the illustrated shuttle lock 18 has been made airtight by the application of a double sealing O-ring on the locking pin 59, which is donned like a garter for sealing of the prosthetic socket 2. A nylon sheath (not illustrated) on the outside of the liner distributes vacuum around the outside of the Hypobaric Prosthetic Liner 3. The nylon sheath works identically like the thin prosthetic sweat wicking sock 5, allowing an even distribution of vacuum over the outside of the liner.

Another observation of the application of both vacuum to the inside of the liner and the outside is that it creates the desirable situation of greater amputee control and feedback from the limb. The application of vacuum to the inside and outside of the liner draws the flexible liner tightly to the walls of the socket, eliminating pistoning or elongation of the liner inherent during swing phase of the amputee's gait. This configuration can be used on above-knee, below-knee and upper-extremity amputees.

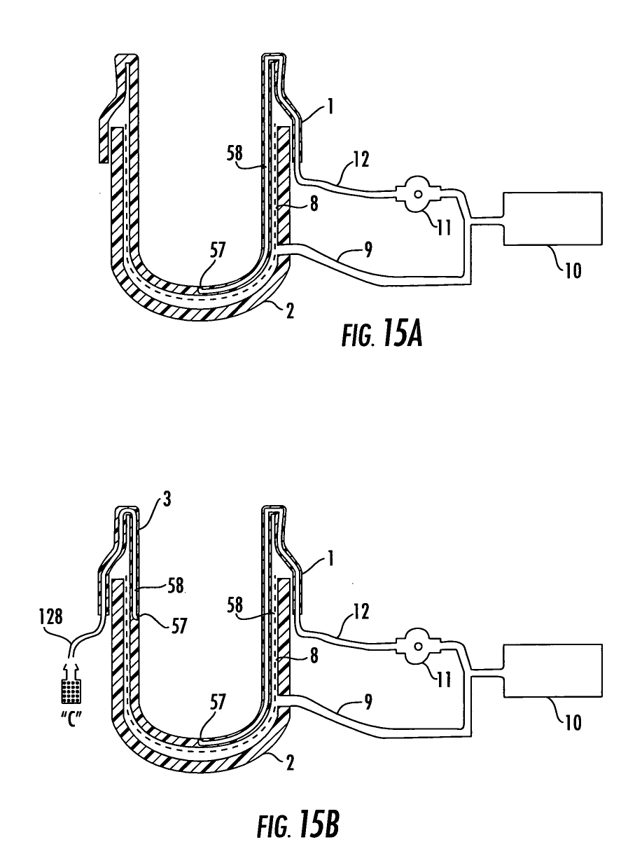

FIGS. 15A and 15B illustrate embodiments of the apron/curtain applied to the Hypobaric Prosthetic Liner that has embedded vacuum lines 58, delivering vacuum to the inside of the prosthetic liner 3 and/or atmospheric air into the liner as well. The liner and socket design in FIG. 15A is outfitted with an internal vacuum passageway 58 passing though the body of the liner and the apron/curtain 1. It is also outfitted with an internal vacuum port 57. The vacuum tubing 12 connects to an adjustable vacuum regulator 11. The vacuum tubing line 12 is branched off the main vacuum line 9 to the prosthetic socket 2 and the outside surface area of the liner 3. The vacuum source 10, is a commercially available vacuum pump designed to deliver vacuum to the outside of the prosthetic liner.

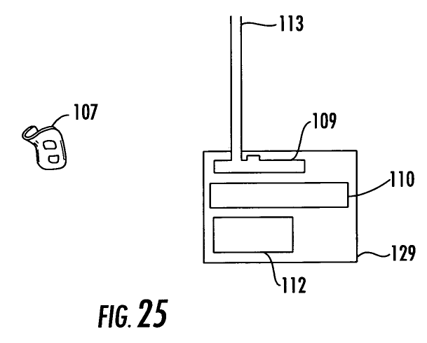

The liner and socket design of FIG. 15B has dual internal tubing 58 passing through the hermetically bonded apron/curtain. It has a second internal vacuum passage way and a second vacuum port. A push button insert, attaching to the external tubing 128 allows air to inflow into the liner, as needed, to cool the limb. This insert would be best used with a commercially available battery powered vacuum pump 10. A remote control solenoid valve as depicted in FIG. 25 could also be attached to the external tubing 128, which would allow air to inflow and cool the limb on demand.

An electronic vacuum regulator may be necessary with liners and limbs configured as illustrated in FIG. 15B. Although not currently illustrated, an electronic vacuum regulator would overcome the potential mass flow limitations of the manually adjustable vacuum regulator 11. The mass flow limitations only becomes an issue in designs that have a relief valve in that an open flow of air is needed for cooling and traditional vacuum regulators restrict flow. A solenoid valve in conjunction with a vacuum sensor and driver board overcomes the mass flow restrictions of typical vacuum regulators. In both versions a nylon or prosthetic sock 8 is on the outside of the prosthetic liner 3 and distributes negative pressure to suspend the prosthetic on the liner. This configuration can be used on above-knee, below-knee and upper-extremity amputees.

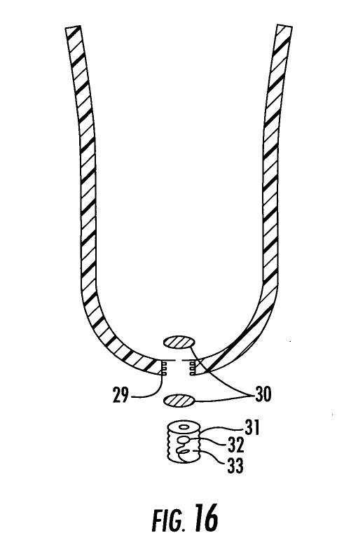

Depicted in FIG. 16 is an alternative embodiment of the Hypobaric Prosthetic Liner which employs a constant differential pressure mechanism 31 to regulate vacuum to the inside of the prosthetic liner. This constant differential pressure mechanism/vacuum regulator 31 is inserted, molded or screwed into the bottom 29 of the liner. A seated poppet 32 and spring 33 is how the vacuum is regulated. Different springs, of different calibrated tensions are employed to create a range of differential pressures between the inside and outside of the liner. The cracking pressure of this regulator (basically this is check valve employed as a pressure regulator) equals the constant differential pressure between the vacuum outside the liner and inside the liner. For example if a regulator/check valve was employed with a spring such that the cracking pressure was 17″Hg, and the outside vacuum being applied to the liner was 20″Hg then the inside of the liner will be 3″Hg. This embodiment is only employed in limbs that have both vacuum applied to inside and outside of the liner. If low tension springs were employed, a traditional suction suspension with expulsion valve could be employed. Spring tension may be regulated by a mechanism to compress the spring and thus increase the cracking pressure.

Although not illustrated, a thin prosthetic sock is employed in this embodiment between the stump and the liner. Filtering screens 30 protects the check valve from clogging. Mineral deposits from perspiration are also filtered by the prosthetic sweat sock. Another feature of this vacuum regulator design will be the feature to compress or lessen the spring tension of the regulator, which will raise or lower the cracking pressure of the regulator. The sock (not illustrated) is a removable thin ply prosthetic sock that distributes vacuum between the inside of the liner and the stump. This configuration can be used on above-knee, below-knee and upper-extremity amputees.

In the prior art there is an example of a prosthetic liner fitted with a check valve. U.S. Pat. No. 6,544,292 Laghi describes a device as PROSTHETIC LINER WITH INTEGRAL AIR EXPULSION VALVE. This patent depicts a prosthetic liner with an air expulsion valve built into the walls of the liner, to facilitate donning of the limb and creating an airtight seal upon the limb. There is no mention of perspiration removal or its application with elevated vacuum. There is also no prosthetic sock donned between the limb and liner in Laghi's design so that air expulsion would not be as effective, or complete as if used with a prosthetic sock.

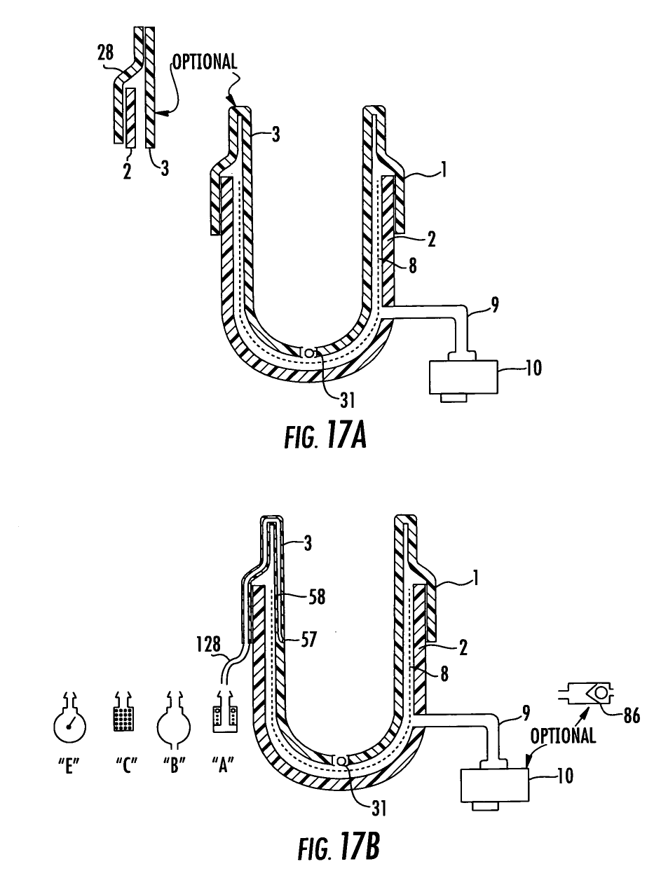

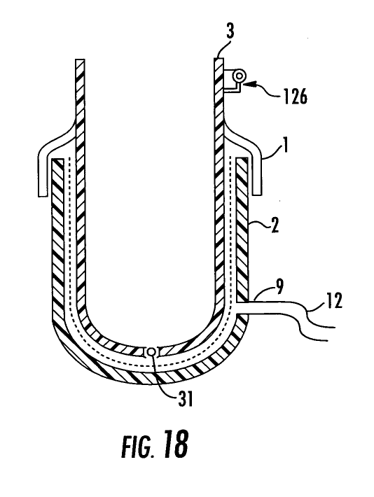

FIGS. 17A and 17B represent an alternative embodiments of the Hypobaric Prosthetic Liner 3 that employs a differential pressure mechanism 31, creating a constant differential pressure between the vacuum level inside and outside of the liner 3. The physical device of this constant differential mechanism is a check valve outfitted with springs of different tensions to create the pressure differential. The placement of this constant differential mechanism 31 can be anywhere on the liner that touches the thin prosthetic sock (not illustrated). As illustrated it is screwed into the bottom of a prosthetic liner. Many prosthetic liners have a 10 mm thread distally to receive a suspension pin. The constant differential pressure mechanism can be adapted to fit into this threaded insert, with or without an accompanying silicone umbrella to adjust for the varying depth of threads found in different prosthetic liners. The cracking pressure of the valve, which is adjustable, is the source of differential pressure necessary for perspiration removal and all the other stated benefits of drawing vacuum to the inside of the liner. In this configuration, elevated vacuum, being supplied by a prosthetic vacuum pump initiates the evaporation of perspiration. Sweat is turned into vapor and removed.

Illustration FIG. 17A shows the optional custom suspension sleeve 28 as well as the apron/curtain 1 configuration. A commercially available prosthetic vacuum pump 10 creates a vacuum down the flexible tubing 9 to the vacuum socket 2. A nylon or sock 8 distributes vacuum to the outside of the liner. The constant differential pressure mechanism 31 allows regulated vacuum to the inside of the liner, between the limb and sock of the user.

Illustration FIG. 17B shows a similarly outfitted liner and socket with the optional expulsion valve 86 to the vacuum pump 10. If low enough spring tensions were employed in both valves 31, 86 the liner would adhere tightly to the limb wearing a prosthetic sock. The sock assists in removing all the dead air space inside the liner and thus elevates the vacuum level on the inside of the liner. An optional fitting attached to the flexible hose 128 would be the vacuum gauge “E” or possibly the push button vacuum relieve valve “C” in this setup. If the vacuum pump was employed, depending on its configuration (body powered, battery powered, or even a reservoir system) different attachments would be inserted into the flexible hose 128, contiguous to the internal vacuum passageway 58 and vent to the internal vacuum port 57. A prosthetic sock distributes vacuum over the stump (not illustrated) and out through the constant differential mechanism 31 to the external nylon or sock 8 down the flexible tubing 9 and expelled by the vacuum pump 10 or expulsion valve 86. This configuration can be used on above-knee, below-knee and upper-extremity amputees.

Depicted in FIG. 18 is an alternate embodiment of the Hypobaric Prosthetic Liner that employs a sealing curtain/apron 1, which is bonded to the liner 3 and configured around the trim lines of the socket. The vacuum relief valve 126 is included. This configuration can be used on above-knee, below-knee and upper-extremity amputees.

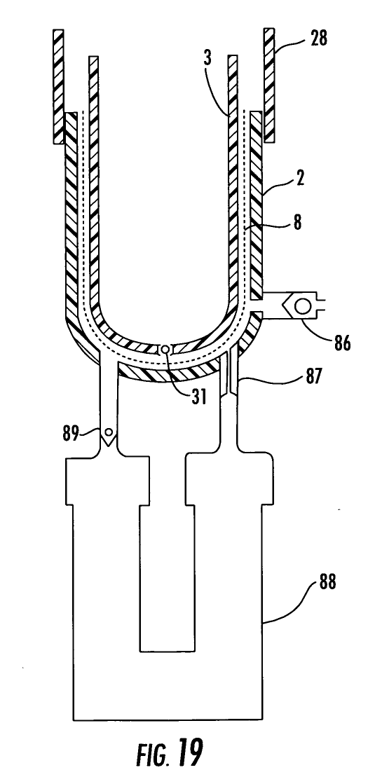

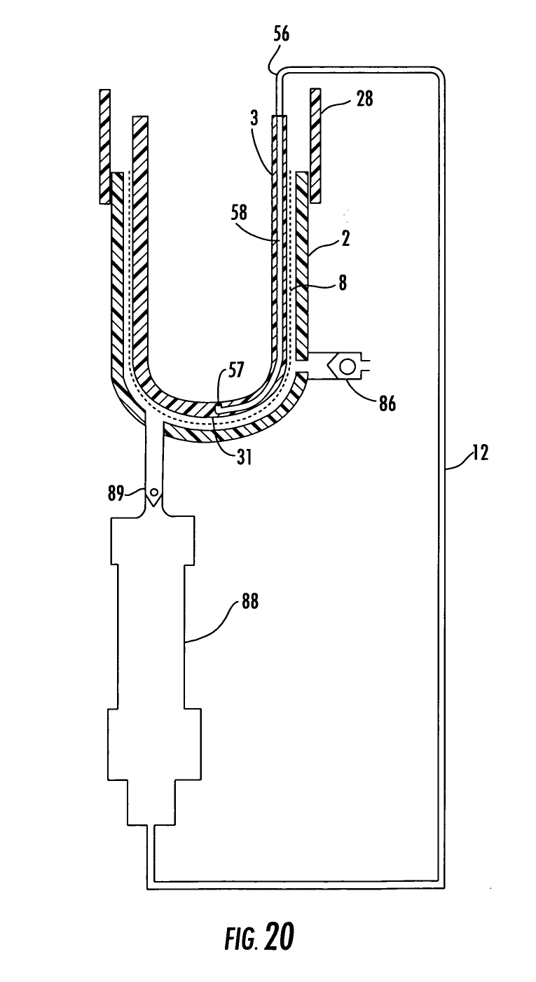

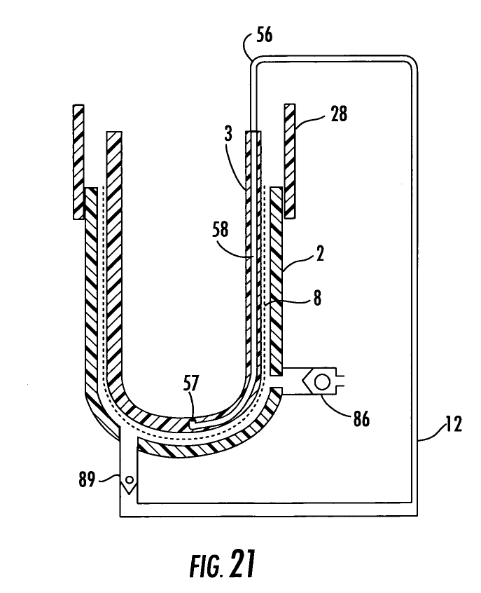

Depicted in FIG. 19 is an alternate embodiment of the Hypobaric Prosthetic Liner, the main component of the Vacuum Assisted Heat/Perspiration Removal System and Limb Volume Management for Prosthetic Device. This embodiment employs vacuum generated from traditional suction suspension and a constant differential mechanism 31. A nylon sheath 8 distributes vacuum over the outside of the liner 3. A custom prosthetic sleeve 28 (or off the shelf) seals the socket 2 to the liner 3, as air is expelled from the check valve 86, negative pressure is generated. A reservoir 88 stores the vacuum and a micro drilled orifice 87 pulls a vacuum, without a differential pressure being created by a spring check valve to the outside of the liner and the inside of the socket. The micro drilled orifice is of such a small diameter that air has a tendency to be expelled out the expulsion valve 86 during stance phase of walking and not forced into the vacuum reservoir 88. A check valve 89 prevents air from entering the reservoir 88 as well as allows vacuum to be drawn onto the outside of the liner 3. A constant differential valve 31 in the liner 3 is optional in this configuration. A sweat wicking prosthetic sock (not pictured) distributes negative pressure over the stump of the amputee. As depicted, the constant differential valve has a low cracking pressure designed to accommodate the limited amount of vacuum generated by this system. It should be noted that hybridization of this design can be achieved with the interchangeable application of an apron/curtain as presented earlier in this application. This configuration can be used on above-knee, and below-knee amputees.

Depicted in FIG. 20 is an alternate embodiment of the Hypobaric Prosthetic Liner that dispenses with the micro drilled orifice and constant differential valve built into the liner. The primary difference in this depicted design is that the reservoir 88 connects directly to the prosthetic liner 3 at the proximal vacuum port 56. With this exception, all functions are identical to FIG. 19. To review, a nylon sheath 8 distributes vacuum over the outside of the liner 3. A custom prosthetic sleeve 28 (or off the shelf) seals the socket 2 to the liner 3, as air is expelled out of the socket via the check valve 86, negative pressure is generated. A reservoir 88 stores the vacuum and a check valve 89 prevents air from entering the reservoir 88. The reservoir is connected to the proximal vacuum port 56 and vacuum is pulled from the internal distal vacuum port 57 up through the internal vacuum passageway 58. A sweat sock (not pictured) distributes negative pressure over the stump of the amputee. It should be noted that this design can be employed interchangeably with an apron/curtain as presented earlier in this application. This configuration can be used on above-knee, and below-knee amputees.

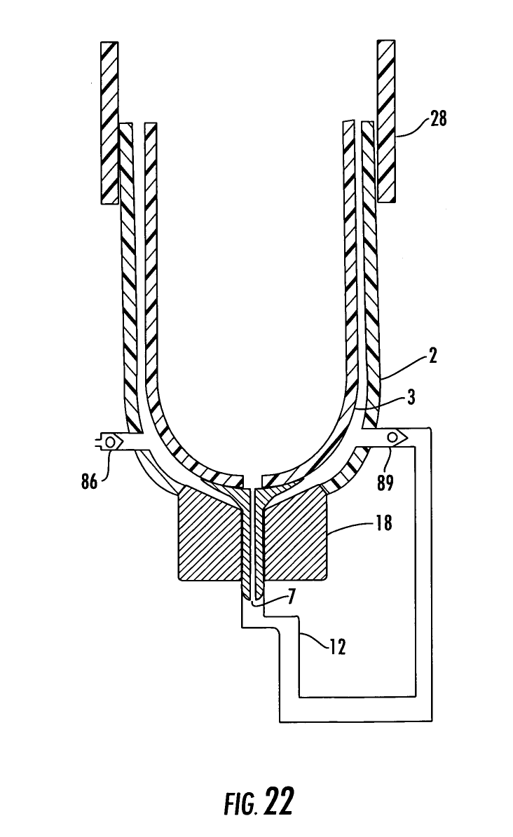

Depicted in FIG. 21 is an alternate embodiment of the Hypobaric Prosthetic Liner 3 that dispenses with the vacuum reservoir. A reservoir allows stored vacuum to be distributed to the limb when the vacuum level drops, allowing for a more even application of vacuum to the limb. The removal of the reservoir has the advantage of simplifying the design and increasing the ease of construction of the artificial limb. This configuration can be used on above-knee, and below-knee amputees.

Depicted in FIG. 22 is an alternate embodiment of the Hypobaric Prosthetic Liner 3 that uses a hollow locking suspension pin to deliver vacuum to the inside of the liner. As illustrated, a prosthetic sleeve 28 seals the socket 92 to the liner 3 and limb. A tapered wedge, a suspension sleeve or even a curtain or apron configuration could also be used in this configuration. An expulsion valve 86 removes air from the socket as the walls of the prosthetic liner expand, like a diaphragm during stance phase. A check valve 89 prevents air from entering into the inside of the prosthetic liner 3. Air is pulled from the inside of the liner down the vacuum port 7 in the hollow suspension pin locked into the shuttle lock 18 and out the passageway 12 through the check valve 89 through the vacuum chamber created by the outside of the liner and the inside of the socket, assisted by a nylon sheath or prosthetic sock (not pictured) and out the expulsion valve 86. This configuration can be used on above-knee, and below-knee amputees.

FIG. 23 depicts a tiny 10-32 threaded umbrella 94 that is bonded into the liner, and allows a hose barb to be screwed and sealed to the liner so that vacuum can pass through. For example, in FIG. 2, the flexible hose 113 could be attached with a hose barb screwed into this insert bonded inside the liner (as opposed to just gluing the tubing to the liner). The top of the umbrella 94 has a curved shape and the front has radial holes around the threaded insert to facilitate bonding into the silicone liner. The umbrella can made from any material such as a machined plastic or a cast flexible urethane. It is one of the options available to assist in delivering vacuum to the inside of the prosthetic liner.

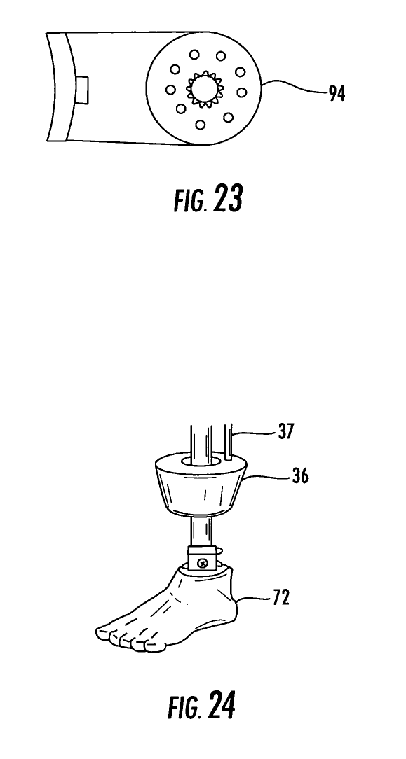

FIG. 24 depicts an alternate embodiment of the battery powered vacuum pump enclosure 36 (as compared to 106 in FIG. 2). The illustration shows one vacuum tube 37 and a prosthetic foot for reference. The battery powered vacuum pump encircles the pylon for easy modular installation in an endoskeletal prosthetic limb.

FIG. 25 illustrates a remote control actuated vacuum relief valve system that has a battery power source 112, a low power receiver/driver board 110 and a magnetically latching solenoid valve 109 housed in an electronics enclosure 129. When a button is pushed on the key chain radio signal sender 107, (which has its own enclosed battery power source), the latching solenoid valve opens and air is allowed to pass into the prosthetic liner via the flexible tubing vacuum passageway 113. Depressing the other button on the keychain sender 107 closes the valve and air flow is ceased. This embodiment is similar to the vacuum pump system presented in FIG. 2. There are certain advantages to putting the solenoid valve into a separate packaged system. Doing so allows even further hybridization of the designs presented in this application. It allows the use of biomechanical powered vacuum pump systems as presented in FIGS. 5 and 6 as the vacuum source with the above RC regulated air inflow and vacuum relief valve system. The solenoid valve 109 is ported in such a way that during full air flow through the system, a maintained level of vacuum is retained on the inside of the Hypobaric Prosthetic Liner. This radio controlled solenoid vacuum relief valve 109 could be an additional insert as presented in FIGS. 9, 12C, and 26. This device could be also used in preexisting vacuum limbs that already have a battery powered vacuum source (or body powered), so that the addition of the Hypobaric Prosthetic Liner with a remote control solenoid valve, could be retrofitted into such limbs.

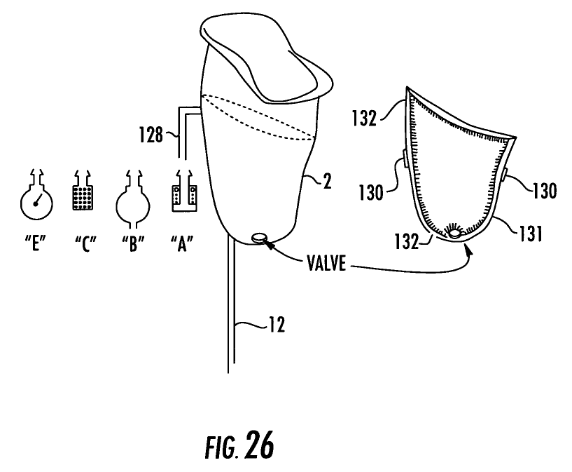

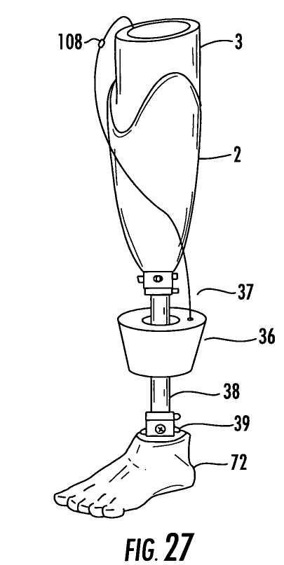

FIG. 26 depicts an above knee artificial limb socket 2 outfitted with perspiration control and elevated vacuum. In its simplest description, a prosthetic sock is bonded to the inside of a traditional rigid Dacron above knee socket and a vacuum is drawn upon the sock, which provides a wicking action over a large area of the limb. Although prosthetic liners are used in all levels of amputation, it is advantageous to have the skin of the AK limb distracted with a pull sock when donning a limb. This creates a spring effect, locking the prosthetic limb onto the patient because the tissues retract creating an air tight seal. This effect has never been duplicated by placing a prosthetic liner on a limb of an above knee amputee. Sometimes it is just difficult to don a liner on to an above knee limb because of the laxity of the tissue. This embodiment provides the benefits of elevated vacuum suspension to a traditional above knee socket. Although a rigid socket is illustrated, this technique could be adapted to the flexible inner socket technique of above knee limbs as well.

A rigid sock frame 131 made from thin PETG or laminated Dacron or urethane is created so that a thin sock can be adhered to it and folded over its proximal edges. The hole is cut out for the expulsion valve and the sock pulled through and reflected and glued around the exterior of the hole. Hook and loop suspend the rigid insert 131 into the socket 2. Other suspension techniques could be employed to give a secure purchase for the insert as well as facilitate its removal. A pin lock or a lanyard system could be employed to secure the rigid sock frame 131. Given careful fabrication techniques the structure becomes monolithic inside the socket without any noticeable ridges that may abrade the stump of the amputee. A vacuum source is attached to the vacuum tubing 12 and connected to the socket. Vacuum ports 132 in the rigid structure 131 are reinforced with mesh screens to prevent window edema. A series of inserts (described in FIG. 9) attached to the flexible hose vacuum passageway 128 achieve different functions and dynamic conditions in this embodiment. The remote control solenoid valve illustrated in FIG. 25 is another possible hybridization of the design when inserted into the vacuum passageway 128. Although an above knee is illustrated, it is possible for this embodiment to be adapted to other levels of amputation.

FIG. 27 is post operative limb that employs the regulated vacuum technology detailed herein. An immediate post operative prosthesis (IPOP) or an early post operative prosthesis (EPOP) is supplied to patients after amputation. There is a substantial body of literature that supports the therapeutic effects of early ambulation after amputation. The benefit of applying regulated vacuum to the inside of a prosthetic liner 3 on a recently amputated limb may include assisting in healing of the wound, shaping of the limb and wound drainage. The limb is covered with an antimicrobial prosthetic sock and covered with an appropriately sized prosthetic liner 3 and is fitted into an adjustable prosthetic socket 2 or temporary fiberglass cast on the limb. An internal line of vacuum is in communication with the prosthetic sock distributing vacuum over the recently amputated limb. Flexible tubing 37 connects to vacuum pump 36 that is outfitted with a removable filter for potential wound drainage. Endoskeletal components complete the post operative limb with an adjustable pylon 38 (by cutting), an adjustable tube clamp adapter 3), and a prosthetic foot 72. Although not pictured, a suspension sleeve is employed so that suspension is afforded. It should be pointed out that the many embodiments of distributing vacuum to the inside of the prosthetic liner detailed herein can be used in hybridization of the post operative limb. Although a vacuum relief mechanism is not illustrated, it could be added to the design.

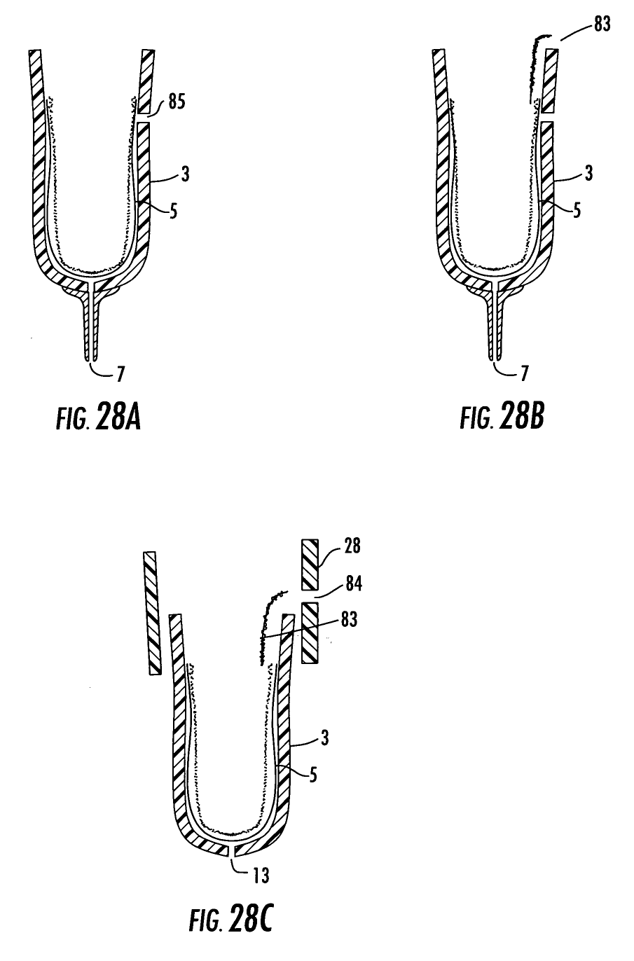

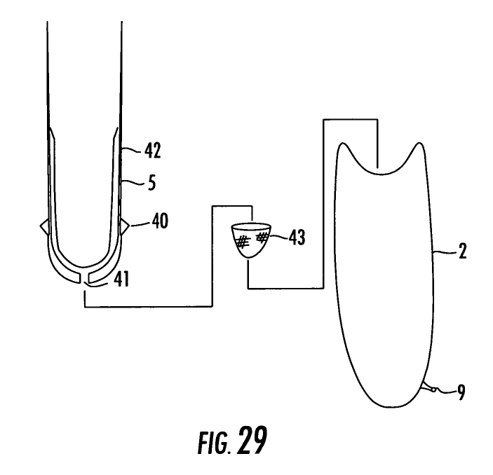

Depicted in FIGS. 28A-28C are embodiments allowing free flow of air through the Hypobaric Prosthetic Liner. The embodiments of FIG. 28A, 28B (when wick is extended) and 28C do not seek to create a closed system of vacuum between the skin and liner 3. These designs allow vacuum to push air through the liner, which in effect maintains a level of vacuum in the system as long a the pump is generating sub-atmospheric pressure. Vacuum level is dependent on air flow, so the size of the inlet hole or mechanism will maintain a vacuum level insside the liner 3.

A prosthetic socket with liner 3 is an excellent insulator (or a poor conductor of heat). Heat and moisture are an excellent environment for bacteria. These designs address this problem by allowing air to free flow through the liner 3 and thus cool the limb.

Illustration 28A depicts a pin lock prosthetic liner 3 with a vacuum port 7 through the suspension pin. There is a sweat sock 5 that acts to wick and distribute the air flow. The surface area/micro structure of the wetable sweat sock 5 promotes evaporation of moisture. Water being evaporated is an endothermic reaction where heat is absorbed. A proximal air port 85 is unregulated and allows air to flow into the liner when drawn by a vacuum source (body powered would be most practical for this application). The sweat sock allows air to flow over the limb out to the distal vacuum port 7.

Illustration 28B depicts a pin lock prosthetic liner 3 with a vacuum port 7 through the suspension pin. An air wick 83, which draws in atmospheric air when positioned above the proximal aspect the liner, is in physical contact with the sweat sock 5. This configuration allows for a free flow of air through the prosthetic liner. This setup can also be used in systems that maintain vacuum inside the liner as well as free flow of air through the liner (as depicted in FIG. 4 and FIG. 6). The wick 83, a piece of yarn or a sewn strap connected to the sweat sock 5 can be tucked below the proximal aspect of the liner 3, to prevent air flow through the liner and close the system. Different thicknesses of yarn can be used to regulate the amount of air flow (and vacuum level in the liner). This design is effective in a vacuum source that is battery operated. Although a pin suspended liner is pictured, any liner design that has a port to vent air and moisture can be employed in the design.

Illustration 28C depicts the use of sleeve with an air vent. A prosthetic curtain or apron configuration could also be used here. This illustration shows that the design is not limited by the use of a prosthetic apron/curtain or a sealing sleeve. What is not pictured is the prosthetic socket that has a vacuum source attached. The prosthetic sleeve 28 adheres to the limb of the amputee and the proximal aspect of the rigid socket. Allowing the mechanical adhesion of the prosthetic liner to suspend the limb, air could be introduced inside the liner at the air vent 84 in the prosthetic suspension sleeve and pulled through the wick 83 and sweat sock 5 by a vacuum source (e.g. body powered would be most efficient). Regulation of the amount of air flow and retained vacuum level inside the open system will be a function of the size of the air vent 84. This configuration can be used on above-knee, below-knee and upper-extremity amputees.

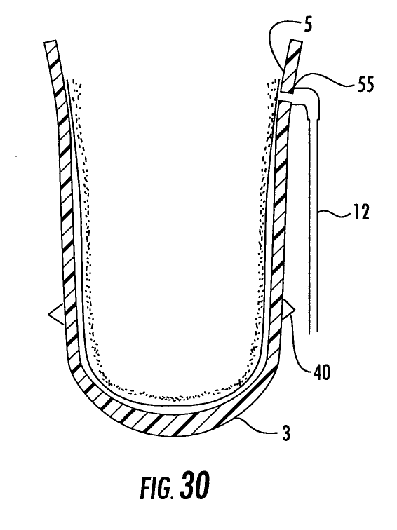

Referring to FIG. 29, this embodiment relates to Ossur's “Seal In” liner which is commercially available. The “Seal In” liner employs a Hypobaric Sealing Membrane (HSM) 40 that creates a vacuum seal between the liner and the socket 2 and thus suspends the limb. A company called Daycor also has a product out that uses a Hypobaric sealing membrane. The embodiment illustrated has a unique vent hole/vacuum port 41 below the Hypobaric Sealing Membrane, so that elevated vacuum applied through the socket 2 at the vacuum tube 9 will allow vacuum to be distributed over the distal limb via the thin prosthetic sock 5. The sock 5 as illustrated is a removable thin ply prosthetic sock. A vacuum pump (or even just an expulsion valve) can be connected to the vacuum tubing 9.

“Seal In” liners employ a fabric covering, but the addition of a short nylon sheath or very thin limb sock 43 may be needed to distribute vacuum over the area below the HSM and thus allow vacuum to be distributed through the vacuum port 41 and to the distal limb. Regulation of the vacuum will be achieved by the use of a vacuum regulator if a mechanical pump is employed (or a vacuum sensor of a battery operated vacuum pump). When regulated vacuum is applied to the limb, air is removed from inside the liner and atmospheric pressure holds the skin of the amputee and the inside of the liner in tight contact. Again, it should be pointed out that there is no increase in radial compression of the liner upon the limb because of the application of regulated vacuum. There is, however, a dramatic increase in intimate contact between the liner and the limb. Volume management is maintained in a limb of such configuration because during swing phase, the weight of the artificial limb and angular acceleration pull on the liner which is in tight adherence with the limb, because of the application of vacuum, causing traction of the distal tissues relative to the tibia, which increases the average volume of distal tissue, creating negative pressure inside the limb, resulting in interstitial refill.

It should also be noted that wicking of perspiration is achieved in this alternative embodiment, as well as a cooling effect on the limb which is a result of the vaporization or evaporation of perspiration. A vacuum pump of any particular configuration is uniquely employed in the drawing of vacuum to the inside of the liner for perspiration control, cooling and volume management, and reduction of movement of the liner relative to the distal tissue, as well as the transference of shear from the skin to the prosthetic liner as the skin will be immobilized. It should be noted that any hybridization of this design can be achieved with the various embodiments of the Hypobaric Prosthetic Liner presented. Although a vacuum relief mechanism is not illustrated, this feature could be added to the design. This configuration can be used on above-knee, below-knee and upper-extremity amputees.

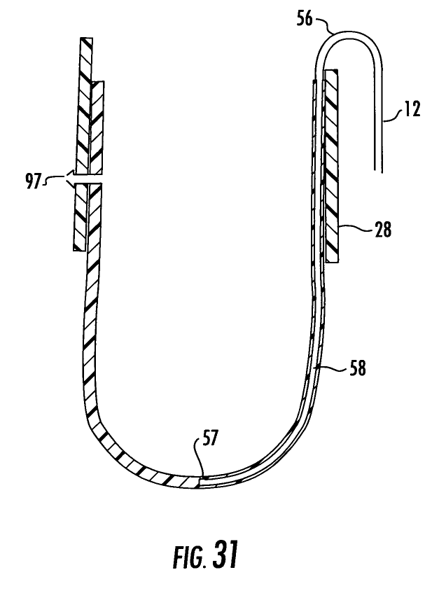

FIG. 30 depicts an alternative approach of delivering regulated vacuum to the inside of the prosthetic liner 3 with a hypobaric sealing membrane 40. A proximal vacuum port 55 is above the trim lines of the rigid prosthetic socket (not illustrated), but not above the level of the prosthetic sweat wicking sock 5, which acts as a wick to distribute negative pressure evenly over the limb. The sweat sock, as illustrated, is a removable thin ply prosthetic sock. The flexible tubing vacuum passageway 12 connects to a vacuum source. A Hypobaric Sealing Membrane 40 is illustrated and this is a member that seals the outside of a prosthetic liner to the inside of a rigid socket. It should be noted that any hybridization of this design can be achieved with the various embodiments of the Hypobaric Prosthetic Liner presented. Although a vacuum relief mechanism is not illustrated, this feature could be added to the design.

Depicted in FIG. 31 is an alternate embodiment of the Hypobaric Prosthetic Liner that employs a sealing hose barb 97 that allows a flexible hose to be connected through a prosthetic sleeve 28, while not allowing air to leak out through the hole. A low durometer o-ring is employed between the sleeve 28 and barb so that the sealing sleeve will not leak vacuum. Other then this sealing hose barb 97 the function is identical to FIG. 9. The illustrated inserts in FIG. 9 could be attached to the hose barb 97 in FIG. 31. This configuration can be used on above-knee, below-knee and upper-extremity amputees.

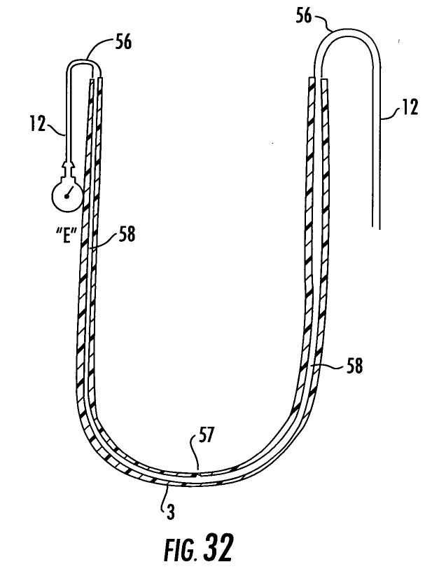

Depicted in FIG. 32 is an alternate embodiment of the Hypobaric Prosthetic Liner 3 that employs a single vacuum passageway 58 that loops through the liner allowing the attachment of a vacuum gauge “E” to one of the two exposed flexible tubing members 12. The other side flexible tubing 12 is ultimately attached to a vacuum source. An internal vacuum port 57 delivers vacuum to the inside of the liner. This design is a modification of the designs illustrated in FIGS. 8 & 9, and can be outfitted with a bonded apron/curtain, sealing sleeve or any desired suspension method. Although a vacuum relief mechanism is not illustrated, this feature could be added (in either proximal or distal planes) to the design.

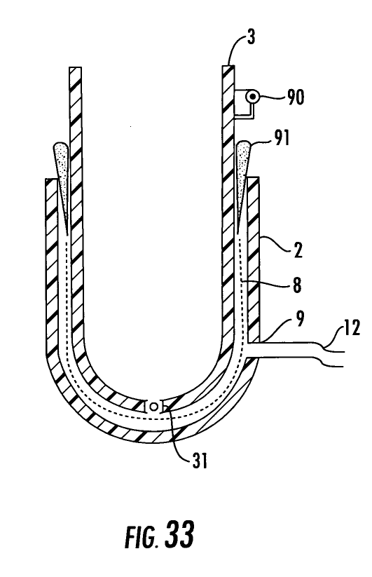

Depicted in FIG. 33 is an alternate embodiment of the Hypobaric Prosthetic Liner. A constant differential mechanism 31 is used to regulate vacuum to the inside of the prosthetic liner 3 from a vacuum environment between the outside of the liner 3 and inside the socket 2. A non-adjustable vacuum relief valve 90 is employed to allow air flow inside the prosthetic liner while maintaining a therapeutic vacuum level inside the liner. A vacuum source is attached to the flexible hose vacuum passageway 12 which allows vacuum to be drawn through the socket 2 to the outside of the liner 3. A nylon sheath 8 distributes vacuum between the inside of the socket 2 and outside of the liner. The constant differential mechanism 31 allows a proportional vacuum level of lesser intensity from the outside vacuum level to form inside the prosthetic liner. Vacuum is distributed over the limb by a sweat wicking prosthetic sock (not pictured).

The non-adjustable vacuum relief valve 90 is set to open at a set vacuum level and as the vacuum level rises from the vacuum source (body powered would be the most efficient in this setup), the differential vacuum level supplied by the constant differential valve 31, eventually raises to a level that opens the vacuum relief valve. This results in the maintenance of a set vacuum level inside the liner while allowing a steam of atmospheric air into the closed system to cool the limb. Air enters into the inside of the liner through the non-adjustable vacuum relief valve 90, travels down a sweat sock (not pictured) out through the constant differential valve 3), is wicked along the nylon sheath 8 through the hose barb 9 connected to the socket 2 and out the hose/vacuum passageway toward the vacuum source (not pictured). A wedge shape gasket 91 is employed in sealing the outside of the liner 3 to the socket 2. The wedge shape is pulled tight under vacuum, and thus increases its sealing ability when exposed to vacuum. This configuration can be used on above-knee, below-knee and upper-extremity amputees.

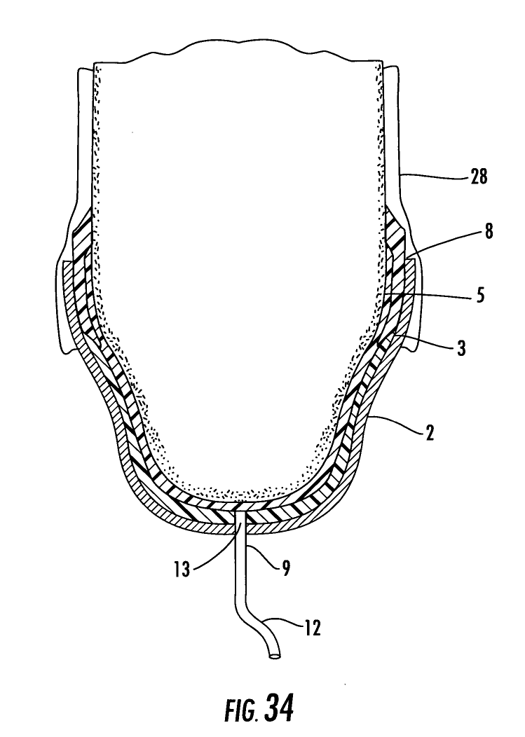

Depicted in FIG. 34 is an alternative embodiment of the Hypobaric Prosthetic Liner 3 that has two proximal vacuum ports 12, 128 that are placed proximally to the trim lines of a prosthetic socket (not illustrated). Depending on the configuration of the vacuum source, various inserts (as illustrated in FIG. 9) are inserted into vacuum port 128 that allow regulated inflow of air into the inside of the Hypobaric Prosthetic Liner 3, distributed by the wicking prosthetic sock 5. Moisture and air are pulled through vacuum line port 12 through the action of the attached vacuum source. A solid locking pin 59 suspends the Hypobaric Prosthetic Liner 3 in the socket. This embodiment can be used on above knee, below knee and upper extremity amputees.

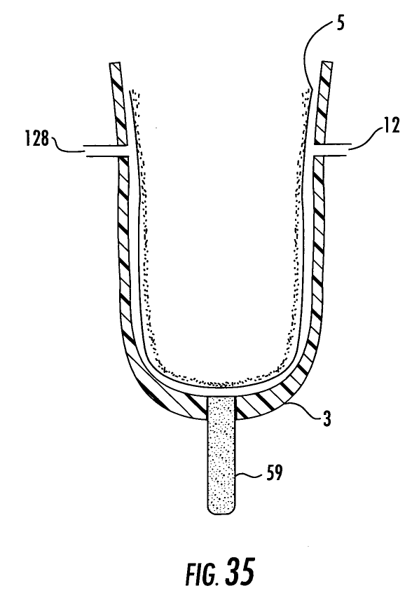

FIG. 35 details an alternate embodiment of the Hypobaric Prosthetic Liner that allows vacuum to be drawn simultaneously to the inside and outside of the prosthetic liner 3. A distal vacuum port/hole 13 in the liner allows fluid communication between a prosthetic sock worn on the limb 5 and a nylon or sock worn the outside of the liner 3. Prosthetic liners without a distal suspension pin are ideal for this configuration. It should be pointed out that the prosthetic sweat sock 5 does not extend proximally above the liner 3. The liner seals to the thigh of the amputee, and a suspension sleeve seals to the proximal socket, liner and above and on up the thigh. A nylon sheath on the outside of the liner acts as a wicking chamber to distribute negative pressure on the outside of the liner, and a sweat wicking sock 5 distributes negative pressure on the inside of the liner via the distal vacuum port/hole creating equal vacuum on both sides of the liner. This allows moisture to be wicked away through the vacuum port 9 that is drilled through the wall of the socket 2, down the flexible tubing vacuum passage way 12 and ultimately to a vacuum source. It should be pointed out that the vacuum passageway ends at the vacuum port 9 in the socket 2 and is not connected in anyway to the liner (as is the case in other embodiments in this text). Although a vacuum relief mechanism is not illustrated, this feature could be added to the design.

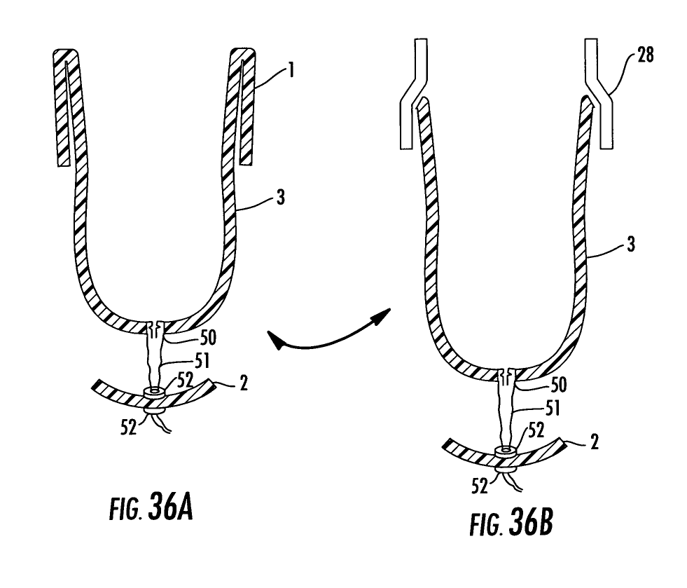

FIGS. 36A and 36B, employ an alternate method of delivering vacuum to the inside of the prosthetic liner 3. A 10 mm threaded recessed 1/16″ hose barb 50 is screwed into the threaded receiver for the traditional pin lock designed liners. This allows a flexible tubing vacuum passageway 51 to be attached at the recessed hose barb 50 and pulled through a sealing grommet 52 and directed outside the rigid prosthetic socket 2, to be ultimately attached to a vacuum source. The sealing grommet can be a captured four lobed O-ring creating an air tight seal on the flexible hose 51. The use of a sealing grommet allows elevated vacuum to be pulled on the outside of the liner if desired or the use of a non elevated vacuum suction suspension employed with the sleeve or apron/curtain 1 or optional illustrated sleeve suspension 2). Although not illustrated, a removable thin ply prosthetic sock is worn between the liner and the limb. This configuration can be used on above-knee, below-knee and upper-extremity amputees. Although a vacuum relief mechanism is not illustrated, this feature could be added to the design.

FIG. 37 depicts an alternative version of the Hypobaric Prosthetic Liner 3 that has a vacuum port 60 along the side of the liner. A sweat wicking sock 5 distributes vacuum between the liner and the limb of the amputee. A specifically modified nylon or prosthetic sock worn on the outside of the liner allows connection of the flexible tubing vacuum passageway 51 to the vacuum port 60. Illustrated in FIG. 37 is a liner that has a pin lock 59 suspension system. There is a hole 61 in the prosthetic socket 2 where the vacuum passageway 51 (flexible hose) is pulled through. The use of a pin suspension allows the hole in the socket not to have an air tight seal. This configuration can also be used any method of securing a prosthetic liner to the socket. Although a vacuum relief mechanism is not illustrated, this feature could be added to the design. This configuration can be used on above-knee, below-knee and upper-extremity amputees.

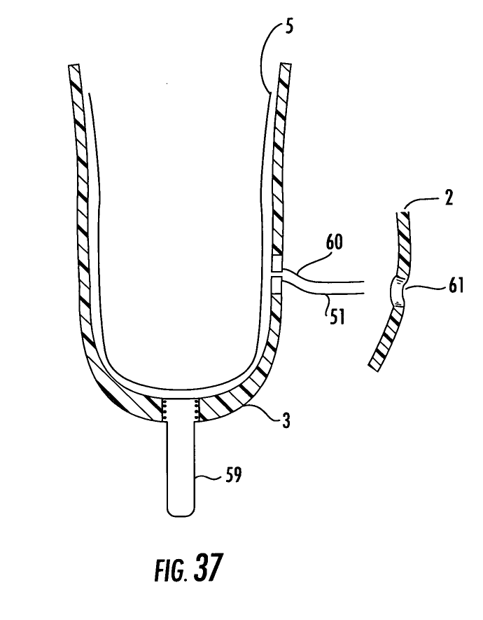

FIG. 38 depicts an alternative embodiment of the Hypobaric Prosthetic Liner 3 that has a sealing grommet 52 that creates an air tight seal on flexible tubing vacuum passageway 51 and the prosthetic socket 2. The flexible tubing vacuum passageway ultimately connects to a vacuum source. The vacuum port 60 is located in the wall of the prosthetic liner 3 and a sweat sock distributes vacuum between the inside of the liner 3 and the sump of the amputee. The sweat wicking sock 5 employed with this design is a removable thin ply prosthetic sock. It should be noted that the design of an integrated breathable fabric or membrane bonded to the inside of the liner 3 could also be employed in the various embodiments of the Hypobaric Prosthetic Liner. It is however desirable to have the ability to remove the sock from the liner for hygienic purposes as it also serves as a filter for the pump system capturing dirt particles and mineral deposits. A specifically modified nylon or prosthetic sock worn on the outside of the liner is constructed to allow connection with the vacuum passageway 51. The sealing grommet can be a captured four lobed O-ring to create an air tight seal so that suction or elevated vacuum could be drawn on the outside of the Hypobaric Prosthetic Liner 3 and inside the rigid socket 2. In such an instance a sealing sleeve or apron/curtain would be employed to seal the system and provide redundant suspension of the solid suspension pin 59. Although a vacuum relief mechanism is not illustrated, this feature could be added to the design. This configuration can be used on above-knee, below-knee and upper-extremity amputees.

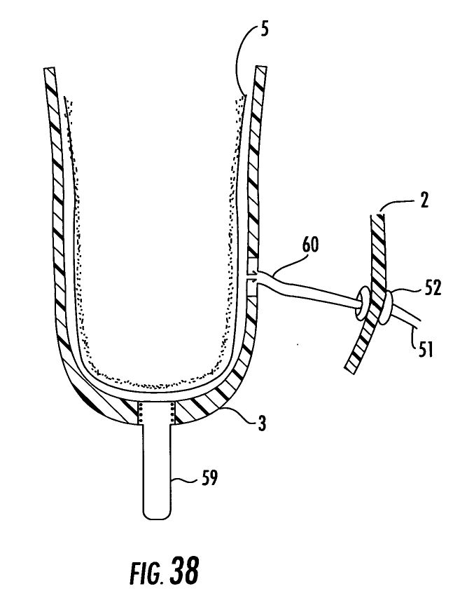

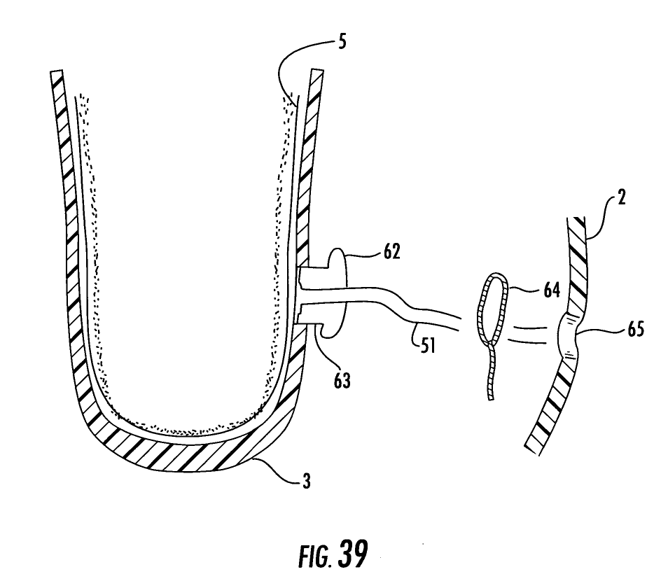

FIG. 39 depicts an alternative embodiment of the Hypobaric Prosthetic Liner 3 that has a sealing button 62 molded into the liner. In the center of the molded button 62 is a vacuum port connected to a vacuum passageway 51 that ultimately connects to a vacuum source. A thin prosthetic sock 5 distributes vacuum between the liner 3 and the limb of the amputee. A specifically modified nylon or prosthetic sock worn on the outside of the liner allows connection of the flexible tubing vacuum passageway 51 to the liner 3. The sealing button 62 is pulled though the rigid socket 2 through a hole in the side of the socket 65, creating an air tight seal.

A pull string 64 is looped over the shoulder of the sealing button 63, fed through the prosthetic socket 2 at the receiving passageway hole 65. The pull string assists in pulling the shoulders of the sealing button through the socket 2 creating an air tight seal. It should be noted that the sealing button is not a suspension mechanism, but only a sealing mechanism. The air tight seal allows the use of a sealing sleeve, or bonded apron/curtain to suspend the prosthetic device, with an expulsion valve or even elevated vacuum to the outside of the prosthetic liner or any other desired suspension mechanism. Although a vacuum relief mechanism is not illustrated, this feature could be added to the design. This configuration can be used on above-knee, below-knee and upper-extremity amputees.

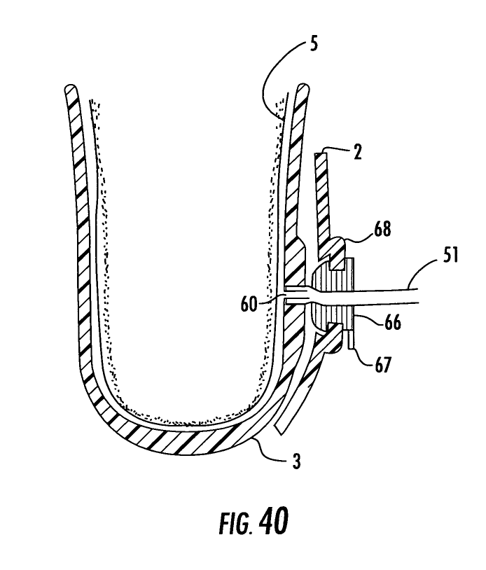

FIG. 40 depicts an alternate version of the Hypobaric Prosthetic Liner that employs a sealing plug that slides down the flexible tubing vacuum passageway 51. The flexible tubing 51 ultimately connects to a vacuum source and the sock 5 acts as a wick, distributing vacuum between the inside of the prosthetic liner 3, sock 5 and limb. The thin prosthetic sock 5 employed with this design is removable and is worn on the inside of the prosthetic liner. A specifically modified nylon or prosthetic sock worn on the outside of the liner will allow for the vacuum tubing 51 to be connected to the liner. The sealing plug creates both an airtight seal on the flexible tubing 51 and to the wall of the socket 2. The air tight seal allows the use of a sealing sleeve, or bonded apron/curtain to suspend the prosthetic device, with an expulsion valve or even elevated vacuum to the outside of the prosthetic liner. The plug is recessed into a pocket 68 in the socket 2 so that it locks into place. A molded flap 67 assists in removal of the sealing plug.

The vacuum port 60 is formed in such a way that the flexible tubing vacuum passageway 51 can be removed from the liner 3 and reattached when the liner in seated in the prosthetic socket 2. A captured four lobed O-ring may be employed in the sealing plug to create an airtight seal on the flexible tubing vacuum passageway 51. Although a vacuum relief mechanism is not illustrated, this feature could be added to the design. This configuration can be used on above-knee, below-knee and upper-extremity amputees.

FIG. 41 depicts an alternate embodiment of the Hypobaric Prosthetic Liner. A threaded receiver 69 accepts a threaded plug 70 creating an airtight seal in the prosthetic socket. The air tight seal allows the use of a sealing sleeve, or bonded apron/curtain to suspend the prosthetic device, with an expulsion valve or even elevated vacuum to the outside of the prosthetic liner. The threaded plug 70 employs an O-ring 71 to facilitate sealing to the threaded receiver mounted on the socket 2. A captured four lobed O-ring may be employed in the threaded receiver to create an air tight seal on the vacuum passageway 51 (flexible hose, most likely 1/16″ ID). The vacuum port 60 is formed in such a way that the vacuum passageway 51 (flexible tubing) can be removed from the liner 3 and reattached when the liner in seated in the prosthetic socket 2.

The vacuum passageway 51 ultimately connects to a vacuum source and the sweat sock 5 acts as a wick distributing vacuum between the inside of the prosthetic liner 3 and the limb. The sweat sock employed with this design is a removable thin ply prosthetic sock. A specifically modified nylon or prosthetic sock worn on the outside of the liner should be included to allow for the vacuum passageway 51. Although a vacuum relief mechanism is not illustrated, this feature could be added to the design. This configuration can be used on above-knee, below-knee and upper-extremity amputees.

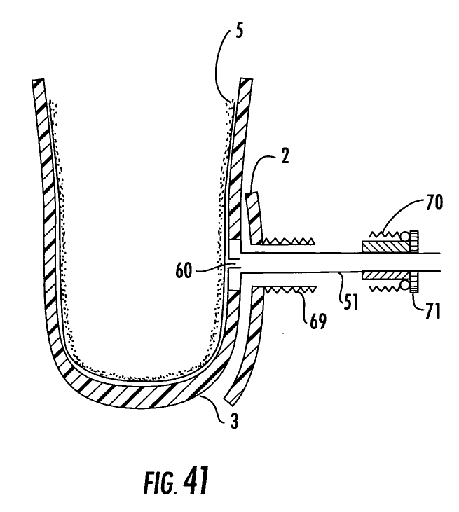

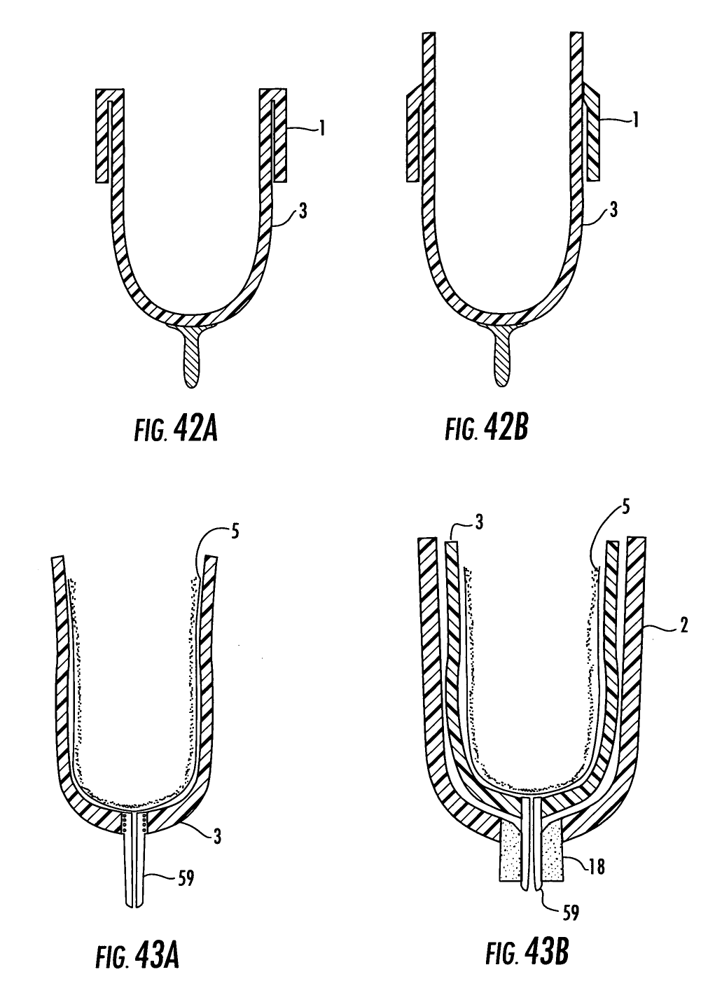

Depicted in FIGS. 42A and 42B are embodiments that employ only the apron/curtain on a prosthetic liner. This embodiment does not have regulated vacuum applied directly to the limb. The illustrated pin suspension liner is an optional feature in this embodiment. This embodiment can be supplied with or without a pin suspension feature. Both versions illustrate the different placement of the vacuum sealing and suspension apron or curtain 1 on the liner 3. The FIG. 42A illustration shows a proximal placement of the apron or curtain bonded to the edge of the liner 3. The FIG. 42B illustration shows a placement of the apron or curtain that is above and follows the trim lines of the socket. A molded, bonded or temporarily adhered curtain/apron 1 provides an airtight seal to the prosthetic socket. This design could be used with limbs that employ elevated vacuum to the outside of the liner or just traditional suction suspension which would involve an air expulsion valve (non elevated vacuum). This configuration can be used on above-knee, below-knee and upper-extremity amputees.

FIG. 43 illustrates the basic components of a hollow suspension pin 59 liner 3. Suspension of the Hypobaric Prosthetic Liner 3 outfitted for pin suspension in the prosthetic socket 2 is solely a mechanical interface of hollow locking pin 59 and shuttle lock 18. The shuttle lock secures the pin via many different methods and releases the pin with a push button mechanism (not illustrated). The advantage of using a pin suspension is the ease and simplicity of the configuration as well as the increased range of knee motion achieved with the removal of the traditional suspension sleeve or other sealing mechanisms. In pin-lock systems, a problematic phenomenon known as pistoning occurs. Pistoning is when distal distraction of the liner occurs because of the weight of the prosthetic limb, which is borne on the distal locking pin. This can cause discomfort, edema, skin irritation and abrasion. When regulated elevated vacuum is applied to a pin lock prosthetic liner, pistoning is minimized, the skin is immobilized, transferring loads and shear from the skin to the liner.

A double wall kit, for example as would be appreciated by those skilled in the art, can be used for this specific application. The Double Wall Kit can be assembled so that it brings vacuum from a source outside the socket 2, to inside the Hypobaric Prosthetic liner 3. The kit, e.g. from Otto Bock, supplies a hollow locking pin, means for making an air tight shuttle lock by employing a double sealing O-ring against a smooth (and hollow) pin and an attachment plate ported for vacuum. The attachment plate in this double wall kit is configured in such a way as to create a clear air passageway to a hose barb outside the rigid socket 2. In this embodiment, the socket attachment plate is used to deliver vacuum to the inside of the Hypobaric Prosthetic Liner 3.

An illustration of such a double wall kit can be found in U.S. Pat. No. 6,926,742 to Caspers described as PLATE/SOCKET ATTACMENT FOR ARTIFICIAL LIMB VACUUM PUMP. It is important to note that this patent only teaches the delivering of vacuum to the inside of a rigid socket and not to the inside of a prosthetic liner containing a prosthetic sock and limb.

The Vacuum Assisted Heat/Perspiration Removal System and Limb Volume Management for Prosthetic Device design is not constrained to the pin lock suspension as evidenced by the embodiments herein. The liners presented can be custom configured to a patient's limb measurements or have standard sizing regarded as “off the shelf” liners. One advantage of a custom prosthetic liner 3, in whatever embodiment, is that it does not have to have a uniform wall thickness. Specific buildups in regions that are sensitive on the limb can be molded in the liner. The fibular head, the distal tibia and tibial crest all have received additional liner material, achieving extra cushion for specific anatomical areas. Weight reduction of the liner is achieved by not having a uniform thick prosthetic liner. These embodiments can be used on above-knee, below-knee and upper-extremity amputees.

Referring to FIG. 44, the sweat wicking prosthetic sock 5 employed in the Vacuum Assisted Heat/Perspiration Removal system and Limb volume management for Prosthetic Device design has features suited for use between the skin and the liner. A sweat wicking sock can refer to any combination of socks inside the prosthetic liner, including a commercially prosthetic sock available from Knit Rite, Inc. named “Liner Liner.”

The illustrated sweat sock has a tapered proximal ply of material beginning at the transition line 19. The reduced ply of material found in the proximal band creates a gently tapered shape. The top of the liner 21 is sewn with a smooth elastic selvage to prevent fraying of the thin ply material. An alternative to the selvage line is a 0.75 inch band of silicone applied to the top of the sweat wicking sock 5, beginning at the transition line 19 and extending approximately 0.75 inch to the top of the sock 21 which helps to control rolling or curling of the proximal aspect of the sock. This top silicone band can be cut in a serpentine fashion to afford extra protection to the skin of the amputee. The added protection of a silicone band is more important to the designs that have elevated vacuum applied to the inside of the prosthetic liner as slightly higher vacuum levels are required to achieve cooling and perspiration removal in such configurations. With the higher vacuum, the more critical is the smooth interface of the prosthetic sock 5 inside the liner.

The purpose of the tapered proximal ply or silicone band is to create a smooth transition between the limb, the sweat sock and the Hypobaric Prosthetic Liner. The smooth transition ensures that the delicate skin of the amputee's limb will not be abraded or damaged by the sweat wicking sock when exposed to regulated vacuum and the dynamic forces created when walking in a prosthetic device. The sweat wicking sock 5 as illustrated is a removable thin ply prosthetic sock. It should be noted that the design of an integrated breathable fabric or membrane bonded to the inside of the liner is also contemplated in the various embodiments of the Hypobaric Prosthetic Liner. It is however desirable to have the ability to remove the sock from the liner for hygienic purposes as it also serves as a filter for the pump system capturing dirt particles and mineral deposits.

Although not illustrated, the sock 5 can also be cut on an angle to cover the knee of a below knee amputee but leave the politeal fold uncovered by the sock. Detrimental wrinkles occur at the back of the knee if the sock is lost its elasticity. When wrinkles occur, localized edema occurs and there is a potential for skin damage. Cutting the sock on a angle leaves the politeal fold open, removing the potential for wrinkles.

The Hypobaric Prosthetic Liner, coupled with a prosthetic vacuum pump, effectively eliminates limb volume fluctuations. Therefore, the prosthetic sweat wicking sock performs different functions when employed with the Hypobaric Prosthetic Liner. Although there are commercially available prosthetic socks designed to be worn under a prosthetic liner, the sweat wicking prosthetic sock differs significantly in function. The main function of the prosthetic sweat sock is to act as a wick allowing perspiration to be drawn away from the limb and expelled from the prosthetic device via vacuum. The prosthetic sweat sock also acts as a means of even vacuum distribution as well as a cushion, distributing external localized pressure over a broader surface area of the limb. Again, the prosthetic sock also acts as filter trapping dirt and mineral deposits from perspiration.

FIG. 45 depicts the tapered cone shape of the vacuum passageway 54 in the Hypobaric Prosthetic Liner 3. This particular shape is resistant to closure under the pressure of weight bearing. It also allows the smallest possible hole to touch the sweat sock and limb.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A vacuum assisted liner system for use with a prosthetic device to be attached to a residual limb, the liner system comprising:
a hypobaric prosthetic liner to be donned over the residual limb; and
a porous wicking material layer to surround at least a portion of the residual limb and define a regulated vacuum environment between the hypobaric prosthetic liner and the residual limb;
the hypobaric prosthetic liner having at least one passageway therethrough defining at least one vacuum port in fluid communication with the regulated vacuum environment.

2. The vacuum assisted liner system according to claim 1, wherein the hypobaric prosthetic liner includes a sealing apron adjacent a proximal end thereof to create a seal between the hypobaric prosthetic liner and at least a portion of the prosthetic device.

3. The vacuum assisted liner system according to claim 1, wherein the porous wicking layer is attached to the hypobaric prosthetic liner.

4. The vacuum assisted liner system according to claim 1, wherein the at least one vacuum port comprises an inlet port in fluid communication with the regulated vacuum environment and an outlet port in fluid communication with the regulated vacuum environment.

5. The vacuum assisted liner system according to claim 4, wherein the at least one passageway comprises a first internal liner passageway connecting the inlet port to the regulated vacuum environment, and a second internal liner passageway connecting the outlet port to the regulated vacuum environment.

6. The vacuum assisted liner system according to claim 4, further comprising a vacuum regulation device connected at least to the outlet port.

7. The vacuum assisted liner system according to claim 6, wherein the vacuum regulation device includes an electric vacuum pump connected to the outlet port, and an associated controller.

8. The vacuum assisted liner system according to claim 7, wherein the vacuum regulation device further includes a flow control switch connected to the inlet port.

9. The vacuum assisted liner system according to claim 8, wherein the flow control switch comprises a solenoid valve.

10. The vacuum assisted liner system according to claim 9, wherein the vacuum regulation device further comprises:
a battery to power the vacuum pump, controller and solenoid valve; and
a housing containing the battery, vacuum pump, controller and solenoid valve.

11. The vacuum assisted liner system according to claim 10, wherein the vacuum regulation device further includes a remote control transmitter for controlling at least the solenoid valve.

12. The vacuum assisted liner system according to claim 6, further comprising a manually adjustable vacuum relief valve connected to the inlet port.

13. The vacuum assisted liner system according to claim 6, wherein the vacuum regulation device includes a motion actuated vacuum pump connected to the outlet port, and an associated mechanical linkage connecting the motion actuated vacuum pump to the prosthetic device.

14. The vacuum assisted liner system according to claim 13, wherein the vacuum regulation device further includes a non-adjustable vacuum relief valve connected to the inlet port of the hypobaric prosthetic liner and in fluid communication with the regulated vacuum environment.

15. The vacuum assisted liner system according to claim 1, further comprising a vacuum regulation device connected at least to the at least one vacuum port; the vacuum regulation device comprising an electric vacuum pump connected to the at least one vacuum port, or a motion actuated vacuum pump connected to the at least one vacuum port.

16. A vacuum assisted liner system for use with a prosthetic device to be attached to a residual limb, the liner system comprising:
a hypobaric prosthetic liner;
a textile fabric layer to define a regulated vacuum environment between the hypobaric prosthetic liner and the residual limb;
the hypobaric prosthetic liner having a plurality of passageways therethrough defining an inlet port and an outlet port in fluid communication with the regulated vacuum environment; and
a vacuum regulation device connected between the inlet port and the outlet port, the vacuum regulation device including an electric vacuum pump connected to the outlet port, and a solenoid valve connected to the inlet port.

17. The vacuum assisted liner system according to claim 16, wherein the plurality of passageways comprise a first internal liner tube connecting the inlet port to the regulated vacuum environment, and a second internal liner tube connecting the outlet port to the regulated vacuum environment.

18. The vacuum assisted liner system according to claim 16, wherein the vacuum regulation device further comprises:
a battery to power the vacuum pump, controller and solenoid valve; and
a housing containing the battery, vacuum pump, controller and solenoid valve.

19. The vacuum assisted liner system according to claim 18, wherein the vacuum regulation device further includes a remote control transmitter for controlling at least the solenoid valve.

20. A method of attaching a prosthetic device to a residual limb, the method comprising:
providing a hypobaric prosthetic liner;
providing a porous wicking material layer to surround at least a portion of the residual limb and define a regulated vacuum environment between the hypobaric prosthetic liner and the residual limb;
the hypobaric prosthetic liner having at least one passageway therethrough defining at least one vacuum port in fluid communication with the regulated vacuum environment; and
providing a vacuum regulation device to connect at least to the outlet port.

21. The method according to claim 20, wherein the at least one vacuum port comprises an inlet port in fluid communication with the regulated vacuum environment and an outlet port in fluid communication with the regulated vacuum environment.

22. The method according to claim 21, wherein the at least one passageway comprises a first internal liner passageway connecting the inlet port to the regulated vacuum environment, and a second internal liner passageway connecting the outlet port to the regulated vacuum environment.

23. The method according to claim 22, wherein the vacuum regulation device includes an electric vacuum pump connected to the outlet port.

24. The method according to claim 23, wherein the vacuum regulation device includes a solenoid valve connected to the inlet port.

25. The method according to claim 24, wherein the vacuum regulation device further comprises:
a battery to power the vacuum pump and solenoid valve; and
a housing containing the battery, vacuum pump, and solenoid valve.

26. The method according to claim 24, wherein the vacuum regulation device further includes a remote control transmitter for controlling at least the solenoid valve.

27. The method according to claim 23, further comprising connecting a manually adjustable vacuum relief valve to the inlet port.

28. The method according to claim 20, wherein the vacuum regulation device includes a motion actuated vacuum pump connected for connection to the outlet port, and an associated mechanical linkage to connect the motion actuated vacuum pump to the prosthetic device.

29. The method according to claim 23, wherein the vacuum regulation device further includes a non-adjustable vacuum relief valve connected to the inlet port of the hypobaric prosthetic liner and in fluid communication with the regulated vacuum environment.

30. The method according to claim 20, wherein the hypobaric prosthetic liner includes a sealing apron adjacent a proximal end thereof to create a seal between the hypobaric prosthetic liner and at least a portion of the prosthetic device.
